(19) United States
(12) Patent Application Publication
(10) Pub. No.: US 2025/0025296 A1
(43) Pub. Date: Jan. 23, 2025

(54) DELIVERY HANDLE ARRANGEMENT FOR PROSTHETIC VALVE DELIVERY SYSTEMS

(71) Applicant: MiRus LLC, Marietta, GA (US)

(72) Inventors: Angela Rothfuss, Marietta, GA (US); Jorge Jimenez, Marietta, GA (US)

(21) Appl. No.: 18/904,796

(22) Filed: Oct. 2, 2024

Related U.S. Application Data
(63) Continuation-in-part of application No. 29/851,277, filed on Aug. 26, 2022.
(60) Provisional application No. 63/541,938, filed on Oct. 2, 2023.

(51) Int. Cl. A61F 2/24

(52) U.S. Cl. CPC A61F 2/2427

(57) ABSTRACT

A delivery handle arrangement and system that facilitates the delivery of an expandable medical device to a treatment site, and wherein the delivery handle arrangement includes a housing having an inner core that extends linearly between a proximal end and a distal end of the housing, and wherein the inner core receives a flexible catheter and a balloon catheter that is disposed coaxially within the flexible catheter; an adjustment mechanism that is configured to control a position of the balloon catheter relative to the flexible catheter, and wherein the adjustment mechanism includes a control knob that has a plurality of threads; and a threaded shaft that is configured to translate linearly within the housing; and a switch that is configured to lock or unlocked the adjustment mechanism.

### REFERENCED APPLICATIONS

The present patent application claims the benefit of U.S. Provisional Patent Application Ser. No. 63/541,938 filed on Oct. 2, 2023 and entitled, “Delivery Handle Arrangement for Prosthetic Valve Delivery Systems”, the disclosure of which is hereby incorporated by reference herein in its entirety and made part of the present U.S. utility patent application for all purposes.

The present disclosure is a continuation-in-part of U.S. application Ser. No. 29/851,277 filed Aug. 26, 2022 and entitled, “Delivery Handle Device”, the disclosure of which is hereby incorporated by reference herein in its entirety and made part of the present U.S. utility patent application for all purposes.

### TECHNICAL FIELD

The present disclosure is directed to prosthetic heart valve replacement system, particularly to devices, systems, and methods for transcatheter delivery of expandable prosthetic heart valves, and more particularly to a delivery handle arrangement for an expandable prosthetic heart valve.

### BACKGROUND OF DISCLOSURE

Many cardiovascular devices such as expandable heart valves are inserted into a patient via the patient's vascular system and then expanded at the treatment site. These devices are typically crimped onto catheter prior to insertion into a patient. Medical devices such as transcatheter aortic valves (TAVs) represent a significant advancement in prosthetic heart valve technology. TAVs bring the benefit of heart valve replacement to patients that would otherwise not be operated on. Transcatheter aortic valve replacement (TAVR) can be used to treat aortic valve stenosis in patients who are classified as high-risk for open heart surgical aortic valve replacement (SAVR). Non-limiting TAVs are disclosed in U.S. Pat. Nos. 5,411,522; 6,730,118; 10,729,543;10,820,993; 10,856,970; 10,869,761; 10,952,852; 10,980,632; 10,980,633; and 2020/0405482, all of which are incorporated fully herein by reference.

A TAV is designed to be compressed into a small diameter catheter, remotely placed within a patient's diseased aortic valve to take over the function of the native valve. Some TAVs are balloon-expandable, while others are self-expandable. In both cases, the TAVs are deployed within a calcified native valve that is forced permanently open and becomes the surface against which the stent is held in place by friction. TAVs can also be used to replace failing bioprosthetic or transcatheter valves, commonly known as a valve in valve procedure. Major TAVR advantages to the traditional surgical approaches include refraining cardiopulmonary bypass and/or aortic cross-clamping and sternotomy that significantly reduces patients' morbidity.

However, several complications are associated with current TAV devices, such as mispositioning, crimp-induced leaflet damage, paravalvular leak, thrombosis, conduction abnormalities, and prosthesis-patient mismatch. These complications are potentially associated with the calcification landscape of the native valve, geometric and mechanical properties of the aortic root, blood biochemistry and coagulability associated with the patient, and concomitant conditions such as hypertension, coronary artery disease, heart failure, etc.

Some limitations of current TAVs that prevent its use in lower risks patients are a) vascular complications from large delivery systems, which necessitates smaller profiles, b) paravalvular leak, c) device mispositioning, and d) device failure. TAVR involves delivery, deployment, and implantation of a crimped, stented valve within a diseased aortic valve or degenerated bioprosthesis. Damage to the leaflet tissue can result in increased calcification and early failure of the TAV.

Prosthetic heart valves that are collapsible can be delivered into a patient via a tube-like delivery apparatus such as a catheter, a trocar, a laparoscopic instrument, or the like, thus avoiding more invasive procedures such as full open-chest, open-heart surgery.

When the collapsed prosthetic valve has reached the desired treatment site in the patient (e.g., patient's heart valve that is to be replaced by the prosthetic valve), the prosthetic valve can be expanded in the treatment site.

In conventional delivery systems for prosthetic heart valves, after the prosthetic heart valve is positioned at the treatment site, the positioning of the prosthetic heart valve may need to be adjusted prior to expansion of the prosthetic heart valve at the treatment site. Many delivery devices can adjust the linear position of the prosthetic heart valve in the treatment site, but cannot both linearly and angularly adjust the prosthetic heart valve at the treatment site. Such angular adjustment can be beneficial to adjust the leaflet system of the prosthetic heart valve in the treatment site to improve the effectiveness of the prosthetic heart valve after deployment at the treatment site.

There therefore is a need for further improvements to the devices, systems, and methods for transcatheter delivery of collapsible prosthetic heart valves.

### SUMMARY OF DISCLOSURE

The present disclosure is directed to prosthetic heart valve replacement, particularly to devices, systems, and methods for transcatheter delivery of expandable prosthetic heart valves, and more particularly to a delivery handle arrangement for an expandable prosthetic heart valve. In particular, the present non-limiting disclosure is directed to a delivery handle arrangement that facilitates the delivery of an expandable medical device to a treatment site (e.g., stent, TAV, heart valve, etc.).

In one non-limiting aspect of the present invention, the delivery handle arrangement includes a housing having an inner core that extends linearly between a proximal end and a distal end of the housing, and wherein the inner core receives a flexible catheter and a balloon catheter that is disposed coaxially within the flexible catheter; an adjustment mechanism that is configured to control a position of the balloon catheter relative to the flexible catheter, and wherein the adjustment mechanism includes a control knob having a plurality of threads; a threaded shaft that is configured to translate linearly within the housing; and a switch that is configured to lock and/or unlocked the adjustment mechanism.

In another and/or alternative non-limiting aspect of the present invention, the delivery handle arrangement optionally includes a flex mechanism that is configured to bend the flexible catheter at a predetermined angle. In one non-limiting embodiment, the flex mechanism includes a flex knob that is coupled to a threaded body, wherein the threaded body is contained within the housing; and wherein a threaded insert that is engaged with the threaded body; and wherein an actuation insert has a crimp band; and wherein a wire member is coupled to the flexible catheter and the crimp band. In another non-limiting embodiment, rotation of the flex knob in a first direction causes the threaded insert and the actuation insert to translate linearly along the threaded body from the distal end towards the proximal end, and wherein the wire member pulls the flexible catheter to bend the flexible catheter at the predetermined angle.

In another and/or alternative non-limiting aspect of the present invention, the delivery handle arrangement optionally includes a flex mechanism includes a flex indicator that has a needle, and wherein the needle is configured to translate along the threaded body to indicate to a user a degree of flex that is present in the flexible catheter. The flex indicator can optionally include numbers, letters or other markings to provide the user additional information on the degree or amount of flex in the flexible catheter.

In another and/or alternative non-limiting aspect of the present invention, the delivery handle arrangement optionally includes a rotational mechanism that is configured to rotate the balloon catheter. In one non-limiting embodiment, the rotational mechanism includes an alignment knob that is coupled to the threaded shaft of the adjustment mechanism; and a gear, and wherein rotation of the alignment knob in a first direction causes the balloon catheter to rotate relative to the flexible catheter.

In another and/or alternative non-limiting aspect of the present invention, the delivery handle arrangement optionally includes a control knob that has a first portion and a second portion, and wherein there is optionally provided a switch that is positioned between the first portion and the second portion. In one non-limiting embodiment, the switch optionally includes a channel having a first stop and a second stop. The first stop optionally has a different width form the second stop (e.g., the first stop has a greater width than the second stop); and a catch that is moveable within the channel. In another non-limiting embodiment, the switch optionally has a substantially circular cross-sectional shape; however, other shapes can be used.

In another and/or alternative non-limiting aspect of the present invention, the delivery handle arrangement is optionally configured that when the adjustment mechanism is locked, the catch is optionally positioned at the second stop of the channel; the switch is optionally positioned near the second portion of the control knob; and the catch is optionally engaged with the plurality of threads. In one non-limiting embodiment, rotation of the control knob in a first direction causes the plurality of threads to engage the threaded shaft and translate the threaded shaft linearly from the proximal end towards the distal end within the housing, and wherein rotation of the control knob in a second direction causes the threaded shaft to translate linearly from the distal end towards the proximal end.

In another and/or alternative non-limiting aspect of the present invention, the delivery handle arrangement is optionally configured that when the adjustment mechanism is unlocked, the catch is optionally positioned at the first stop of the channel; the switch is optionally positioned near the first portion of the control knob; and the catch is disengaged from the plurality of threads. In one non-limiting embodiment, the alignment knob of the rotational mechanism is configured to be pushed so as to advance the threaded shaft towards the distal end, and wherein the alignment knob is configured to be pulled so as to retreat the treaded shaft towards the proximal end.

In another and/or alternative non-limiting aspect of the present invention, the delivery handle arrangement optionally includes a luer tree coupled to the alignment knob. The luer tree optionally includes a flush port; a guidewire lumen that is configured to receive a guidewire; and a balloon inflation port.

In another and/or alternative non-limiting aspect of the present invention, the delivery handle arrangement optionally includes a hypotube that is contained within the inner core. The hypotube is optionally configured to allow flush fluid to flow over the balloon catheter and into the flexible catheter. An O-ring is optionally provided so as to create a sliding hemostatic seal between an inner surface of the inner core and an outer surface of the hypotube.

In another and/or alternative non-limiting aspect of the present invention, the expandable medical device that is used with the delivery handle arrangement is a prosthetic heart valve.

In another and/or alternative non-limiting aspect of the present invention, there is provided a system that delivers an expandable medical device to a treatment site. The system includes a flexible catheter; a balloon catheter that is disposed coaxially within the flexible catheter; and a delivery handle arrangement. In one non-limiting embodiment, the delivery handle arrangement includes a housing having an inner core that extends linearly between a proximal end and a distal end of the housing, and wherein the inner core receives at least a portion of the flexible catheter and the balloon catheter; and wherein an adjustment mechanism is configured to control a position of the balloon catheter relative to the flexible catheter, and wherein the adjustment mechanism includes a control knob that optionally has a plurality of threads; and a shaft (e.g., a threaded shaft, etc.) that is configured to translate linearly within the housing; and wherein a switch is optionally configured to lock and/or unlocked the adjustment mechanism, and wherein the switch optionally includes a channel that has a first stop and a second stop, and wherein the first stop optionally has a different width from the second stop (e.g., the first stop has a greater width than the second stop, etc.); and wherein a catch is optionally moveable within the channel.

In another and/or alternative non-limiting aspect of the present invention, the delivery handle arrangement optionally includes a flex mechanism that is configured to bend the flexible catheter at a predetermined angle. In one non-limiting embodiment, the flex mechanism includes a flex knob that is coupled to a body (e.g., a threaded body), wherein the body is optionally at least partially contained within the housing; and wherein an insert (e.g., a threaded insert) is optionally engaged with the body; and wherein an actuation insert has a crimp band; and wherein a wire member is coupled to the flexible catheter and the crimp band, and wherein rotation of the flex knob in a first direction causes the insert and the actuation insert to translate linearly along the body from the distal end towards the proximal end, and wherein the wire member pulls the flexible catheter to bend the flexible catheter at a predetermined angle.

In another and/or alternative non-limiting aspect of the present invention, the delivery handle arrangement optionally includes a rotational mechanism that is configured to rotate the balloon catheter. In one non-limiting embodiment, the rotational mechanism includes an alignment knob that is coupled to the threaded shaft of the adjustment mechanism; and a gear, and wherein rotation of the alignment knob in a first direction causes the balloon catheter to rotate relative to the flexible catheter.

In another and/or alternative non-limiting aspect of the present invention, the delivery handle arrangement optionally includes an adjustment mechanism, and when the adjustment mechanism is locked, a catch is optionally positioned at the second stop of the channel; and wherein the catch is optionally engaged with an engagement arrangement (e.g., a plurality of threads), wherein rotation of the control knob in a first direction causes the engagement arrangement to engage the shaft (e.g., threaded shaft, etc.) and translate the shaft linearly from the proximal end towards the distal end within the housing, and wherein rotation of the control knob in a second direction causes the shaft to translate linearly from the distal end towards the proximal end.

In another and/or alternative non-limiting aspect of the present invention, the delivery handle arrangement optionally includes an adjustment mechanism, and when the adjustment mechanism is locked, the catch is positioned at the first stop of the channel; and the catch is disengaged from an engagement arrangement (e.g., a plurality of threads, etc.), wherein the alignment knob of the rotational mechanism is pushed to advance a shaft (e.g., a threaded shaft, etc.) towards the distal end, and wherein the alignment knob is pulled to retreat the shaft towards the proximal end.

One non-limiting object of the present disclosure is the provision of a delivery handle arrangement that facilitates the delivery of an expandable medical device to a treatment site.

In another and/or alternative non-limiting object of the present disclosure is the provision of a delivery handle arrangement that includes a) a housing that has an inner core that extends linearly between a proximal end and a distal end of the housing, and wherein the inner core at least partially receives a flexible catheter and a balloon catheter, and wherein the flexible catheter and the balloon catheter is at least partially disposed coaxially within the flexible catheter; b) an adjustment mechanism that is configured to control a position of the balloon catheter relative to the flexible catheter, and wherein the adjustment mechanism includes a control knob that has a connection arrangement (e.g., a plurality of threads, etc.), a shaft (e.g., a threaded shaft, etc.) that is configured to translate linearly within the housing, and a switch that is configured to lock or unlocked the adjustment mechanism.

In another and/or alternative non-limiting object of the present disclosure is the provision of a delivery handle arrangement that optionally includes a flex mechanism that is configured to bend the flexible catheter at a predetermined angle.

In another and/or alternative non-limiting object of the present disclosure is the provision of a delivery handle arrangement that optionally includes a flex mechanism that is configured to bend the flexible catheter at a predetermined angle, and wherein the flex mechanism includes a) a flex knob coupled to a body (e.g., a threaded body, etc.), wherein the body is at least partially contained within the housing, b) an insert (e.g., a threaded insert, etc.) that is engaged with the body, c) an actuation insert having a crimp band, and d) a wire member coupled to the flexible catheter and the crimp band.

In another and/or alternative non-limiting object of the present disclosure is the provision of a delivery handle arrangement that optionally includes a flex mechanism that is configured to bend the flexible catheter at a predetermined angle, and wherein the flex mechanism includes a flex knob coupled to a body (e.g., a threaded body, etc.), and wherein rotation of the flex knob in a first direction causes an insert (e.g., the threaded insert, etc.) and an actuation insert to translate linearly along the body (e.g., a threaded body, etc.) from or near the distal end towards the proximal end, and wherein a wire member pulls the flexible catheter to cause the flexible catheter to bend to one or more predetermined angles.

In another and/or alternative non-limiting object of the present disclosure is the provision of a delivery handle arrangement that optionally includes a flex indicator that is used to indicate the amount and/or a degree of flex present in the flexible catheter.

In another and/or alternative non-limiting object of the present disclosure is the provision of a delivery handle arrangement that optionally includes a flex indicator that optionally includes a needle and/or other visual indicator (e.g., meter, digital readout, etc.) that is used to indicate the amount and/or a degree of flex present in the flexible catheter.

In another and/or alternative non-limiting object of the present disclosure is the provision of a delivery handle arrangement that optionally includes a flex indicator that optionally includes a needle, and wherein the needle translates along the body (e.g., a threaded body, et.) to indicate to a user a degree of flex present in the flexible catheter.

In another and/or alternative non-limiting object of the present disclosure is the provision of a delivery handle arrangement that optionally includes a rotational mechanism that is configured to at least partially rotate the balloon catheter.

In another and/or alternative non-limiting object of the present disclosure is the provision of a delivery handle arrangement that optionally includes a rotational mechanism that is configured to at least partially rotate the balloon catheter, and wherein the rotational mechanism includes a) an alignment knob that is coupled to a shaft (e.g., a threaded shaft, etc.) of the adjustment mechanism, and b) a gear, and wherein rotation of the alignment knob causes the balloon catheter to rotate relative to the flexible catheter.

In another and/or alternative non-limiting object of the present disclosure is the provision of a delivery handle arrangement that optionally includes an adjustment mechanism that is configured to cause linear adjustment of the shaft.

In another and/or alternative non-limiting object of the present disclosure is the provision of a delivery handle arrangement that optionally includes an adjustment mechanism that is configured to cause linear adjustment of the shaft, and wherein the adjustment mechanism includes a control knob that has a first portion and a second portion, and wherein a switch is positioned between the first portion and the second portion.

In another and/or alternative non-limiting object of the present disclosure is the provision of a delivery handle arrangement that optionally includes an adjustment mechanism that is configured to cause linear adjustment of the shaft, and wherein the adjustment mechanism includes a control knob that has a first portion and a second portion, and wherein a switch is positioned between the first portion and the second portion, and wherein the switch includes a) a channel having a first stop and a second stop, wherein the first stop optionally has a different width than the second stop, and b) a catch that is moveable within the channel.

In another and/or alternative non-limiting object of the present disclosure is the provision of a delivery handle arrangement that optionally includes an adjustment mechanism that is configured to cause linear adjustment of the shaft, and wherein the adjustment mechanism includes a control knob that has a first portion and a second portion, and wherein a switch is positioned between the first portion and the second portion, and wherein the switch has a substantially circular cross-sectional shape.

In another and/or alternative non-limiting object of the present disclosure is the provision of a delivery handle arrangement that optionally includes an adjustment mechanism that is configured to cause linear adjustment of the shaft, and when the adjustment mechanism is locked then a) the catch is positioned at the second stop of the channel, b) the switch is positioned near the second portion of the control knob, and b) the catch is engaged with engagement arrangement (e.g., a plurality of threads, etc.).

In another and/or alternative non-limiting object of the present disclosure is the provision of a delivery handle arrangement that optionally includes an adjustment mechanism that is configured to cause linear adjustment of the shaft, and wherein the adjustment mechanism includes a control knob, and wherein rotation of the control knob in a first direction causes the engagement arrangement (e.g., the plurality of threads, etc.) to engage the shaft (e.g., a threaded shaft, etc.) and translate the shaft linearly from or near the proximal end towards the distal end within the housing, and wherein rotation of the control knob in a second direction causes the shaft to translate linearly from or near the distal end towards the proximal end.

In another and/or alternative non-limiting object of the present disclosure is the provision of a delivery handle arrangement that optionally includes an adjustment mechanism that is configured to cause linear adjustment of the shaft, and when the adjustment mechanism is unlocked, a) the catch is positioned at the first stop of the channel, b) the switch is positioned near the first portion of the control knob; and c) the catch is disengaged from the engagement arrangement (e.g., the plurality of threads, etc.).

In another and/or alternative non-limiting object of the present disclosure is the provision of a delivery handle arrangement that optionally includes a rotational mechanism that is configured to at least partially rotate the balloon catheter, and wherein an alignment knob of the rotational mechanism is configured to be pushed to advance the shaft (e.g., the threaded shaft, etc.) towards the distal end, and wherein the alignment knob is configured to be pulled to retreat the shaft towards the proximal end.

In another and/or alternative non-limiting object of the present disclosure is the provision of a delivery handle arrangement that optionally includes a luer tree that is coupled to the alignment knob, wherein the luer tree includes a flush port, a guidewire lumen that is configured to receive a guidewire, and/or a balloon inflation port.

In another and/or alternative non-limiting object of the present disclosure is the provision of a delivery handle arrangement that optionally includes a) a hypotube that is at least partially contained within the inner core, wherein the hypotube is configured to allow flush fluid to flow over the balloon catheter and into the flexible catheter, and b) a sealing arrangement (e.g., an O-ring, etc.) that creates a sliding hemostatic seal between an inner surface of the inner core and an outer surface of the hypotube.

In another and/or alternative non-limiting object of the present disclosure is the provision of a delivery handle arrangement that is used with a prosthetic heart valve.

In another and/or alternative non-limiting object of the present disclosure is the provision of a system that delivers an expandable medical device to a treatment site, and wherein the system includes a) a flexible catheter, b) a balloon catheter that is at least partially disposed coaxially within the flexible catheter, c) a delivery handle arrangement, and wherein the delivery handle arrangement includes i) a housing that has an inner core that extends linearly between a proximal end and a distal end of the housing, wherein the inner core at least partially receives the flexible catheter and the balloon catheter, ii) an adjustment mechanism that is configured to control a position of the balloon catheter relative to the flexible catheter, and wherein the adjustment mechanism includes A) a control knob having a connection arrangement (e.g., a plurality of threads, etc.), and B) a shaft (e.g., a threaded shaft, etc.) that is configured to translate linearly within the housing, and iii) a switch that is configured to lock or unlocked the adjustment mechanism, and wherein the switch includes A) a channel that has a first stop and a second stop, wherein the first stop has a different width than the second stop, and B) a catch that is moveable within the channel.

In another and/or alternative non-limiting object of the present disclosure is the provision of a system that delivers an expandable medical device to a treatment site, and wherein the system includes a flex mechanism that is configured to bend the flexible catheter at a predetermined angle, and wherein the flex mechanism includes i) a flex knob that is coupled to a body (e.g., a threaded body, etc.), and wherein the body is at least partially contained within the housing, ii) an insert (e.g., a threaded insert, etc.) that is engaged with the body, c) an actuation insert having a crimp band; and d) a wire member that is coupled to the flexible catheter and the crimp band, and wherein rotation of the flex knob in a first direction causes the insert and the actuation insert to translate linearly along the body from or near the distal end towards the proximal end, and wherein the wire member pulls the flexible catheter to bend the flexible catheter at one or more predetermined angles.

In another and/or alternative non-limiting object of the present disclosure is the provision of a system that delivers an expandable medical device to a treatment site, and wherein the system includes a rotational mechanism that is configured to rotate the balloon catheter, and wherein the rotational mechanism includes i) an alignment knob that is coupled to the shaft (e.g., a threaded shaft, etc.) of the adjustment mechanism, and ii) a gear, and wherein rotation of the alignment knob in a first direction causes the balloon catheter to rotate relative to the flexible catheter.

In another and/or alternative non-limiting object of the present disclosure is the provision of a system that delivers an expandable medical device to a treatment site, and wherein the system includes an adjustment mechanism, and wherein when the adjustment mechanism is locked then a) the catch is positioned at the second stop of the channel, and b) the catch is engaged with the plurality of threads, and wherein rotation of the control knob in a first direction causes the engagement arrangement (e.g., the plurality of threads, etc.) to engage the shaft (e.g., the threaded shaft, etc.) and translate the shaft linearly from or near the proximal end towards the distal end within the housing, and wherein rotation of the control knob in a second direction causes the shaft to translate linearly from or near the distal end towards the proximal end.

In another and/or alternative non-limiting object of the present disclosure is the provision of a system that delivers an expandable medical device to a treatment site, and wherein the system includes an adjustment mechanism, and wherein when the adjustment mechanism is unlocked then a) the catch is positioned at the first stop of the channel, and b) the catch is disengaged from the engagement arrangement (e.g., the plurality of threads, etc.), and wherein the alignment knob of the rotational mechanism is pushed to advance the shaft (e.g., the threaded shaft, etc.) towards the distal end, and wherein the alignment knob is pulled to retreat the shaft towards the proximal end.

These and other objects and advantages will become apparent to those skilled in the art upon reading and following the description taken together with the accompanying drawings.

### BRIEF DESCRIPTION OF THE DRAWINGS

The accompanying drawings, which are incorporated into and form a part of the specification, schematically illustrate one or more example implementations of the disclosed technology and, together with the general description given above and detailed description given below, serve to explain the principles of the disclosed subject matter, and wherein:

FIG. 1 depicts an exemplary, non-limiting delivery system comprising a non-limiting handle arrangement.

FIG. 2 is a cross-sectional side view of the delivery handle arrangement of FIG. 1.

FIG. 3 is a cross-sectional side view of the delivery handle arrangement of FIG. 1, wherein the delivery handle arrangement is configured in a locked position.

FIG. 4 is a cross-sectional side view of the delivery handle arrangement of FIG. 1, wherein the delivery handle arrangement is configured in an unlocked position.

FIG. 5 depicts another exemplary, non-limiting delivery system comprising a non-limiting handle arrangement.

FIG. 6 is a cross-sectional side view of the delivery handle arrangement of FIG. 5.

FIGS. 7A-7D depict an exemplary, non-limiting embolic capture filter membrane sock configured for use with a non-limiting flex catheter and a non-limiting balloon catheter.

### DETAILED DESCRIPTION OF NON-LIMITING EMBODIMENTS

A more complete understanding of the articles/devices, processes and components disclosed herein can be obtained by reference to the accompanying drawings. These figures are merely schematic representations based on convenience and the ease of demonstrating the present disclosure, and are, therefore, not intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any unavoidable impurities that might result therefrom, and excludes other ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values).

The terms “about” and “approximately” can be used to include any numerical value that can vary without changing the basic function of that value. When used with a range, “about” and “approximately” also disclose the range defined by the absolute values of the two endpoints, e.g., “about 2 to about 4” also discloses the range “from 2 to 4.” Generally, the terms “about” and “approximately” may refer to plus or minus 10% of the indicated number.

Percentages of elements should be assumed to be percent by weight of the stated element, unless expressly stated otherwise.

Although the operations of exemplary embodiments of the disclosed method may be described in a particular, sequential order for convenient presentation, it should be understood that disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed.

For the sake of simplicity, the attached figures may not show the various ways (readily discernable, based on this disclosure, by one of ordinary skill in the art) in which the disclosed system, method and apparatus can be used in combination with other systems, methods and apparatuses. Additionally, the description sometimes uses terms such as “produce” and “provide” to describe the disclosed method. These terms are abstractions of the actual operations that can be performed. The actual operations that correspond to these terms can vary depending on the particular implementation and are, based on this disclosure, readily discernible by one of ordinary skill in the art.

Example implementations of the disclosed technology generally provides a medical device that can includes a valve (e.g., heart valve, TAVR valve, mitral valve replacement, tricuspid valve replacement, pulmonary valve replacement, etc.) and a delivery system for delivering the valve to a treatment site (e.g., in a patient's heart valve, etc.). More particularly, example implementations of the disclosed technology provides a delivery handle arrangement that is configured to more accurately deliver such valves to the treatment site. The delivery handle arrangement includes: (i) a flex mechanism that is configured to facilitate flex catheter passage around various bends or arches (e.g. aortic arch bend) and to adjust coaxial alignment of the crimped valve with the aortic annulus prior to deployment; (ii) an adjustment mechanism that is configured to advance or retract the balloon catheter's axial position relative to the flex catheter; and (iii) a commissure alignment mechanism that is configured to rotate the crimped valve and control the rotational orientation of the implant leaflet attachment points (commissure) relative to the native anatomical. The adjustment mechanism can advantageously transition between a locked position and an unlocked position by way of a switch, depending on the amount of control or precision needed when advancing or retracting the balloon catheter.

In accordance with one non-limiting aspect of the present disclosure, the prosthetic heart valve is not limited to a TAV, but can be mitral valve replacement, tricuspid valve replacement, pulmonary or valve replacement. The prosthetic heart valve includes a radially collapsible and expandable frame and a leaflet structure comprising a plurality of leaflets. The prosthetic heart valve can optionally include an annular skirt or cover member disposed on and covering the cells of at least a portion of the frame. The frame can comprise a plurality of interconnected struts and strut joints defining a plurality of open cells in the frame. The frame is partially (e.g., 1-99.999 wt. % and all values and ranges therebetween) or fully made of a metal material.

In some non-limiting aspects of the present invention, the prosthetic heart valve includes a frame, a leaflet structure supported by the frame, and an optional inner skirt secured to the surface of the frame and/or leaflet structure, and an optional outer skirt that is secured to the frame. The prosthetic heart valve can be implanted in the annulus of the native aortic valve; however, the prosthetic heart valve also can be configured to be implanted in other valves of the heart (e.g., tricuspid valve, pulmonary valve, mitral valve). The prosthetic heart valve has a “lower” end and an “upper” end, wherein the lower end of the prosthetic heart valve is the inflow end and the upper end of the prosthetic heart valve is the outflow end. The metal alloy that is used to partially or fully form the frame of the prosthetic heart valve is configured to be radially collapsible to a collapsed or crimped state for introduction into the body (e.g., on a delivery catheter, etc.) and radially expandable to an expanded state for implanting the prosthetic heart valve at a desired location in the body (e.g., the aortic valve, tricuspid valve, pulmonary valve, mitral valve, etc.). The frame of the prosthetic heart valve can be formed of a plastically-expandable material that permits crimping of the frame to a smaller profile for delivery and expansion of the frame. The expansion of the crimped frame of the prosthetic heart valve can be by an expansion device such as, but not limited to, a balloon of on a balloon catheter; however, the frame can optionally be partially (e.g., 1-99.999 wt. % and all values and ranges therebetween) or fully formed of a self-expanding material (e.g., Nitinol, etc.). The frame at least partially (e.g., 1-99.999 wt. % and all values and ranges therebetween) formed of a plurality of angularly spaced, vertically extending posts, or struts. The posts or struts can optionally be interconnected via a lower row of circumferentially extending struts and an upper row of circumferentially extending struts via strut joints. The struts can be arrangement in a variety of patterns (e.g., zig-zag pattern, saw-tooth pattern, triangular pattern, polygonal pattern, oval pattern, etc.). One or more of the posts and/or struts can have the same or different thicknesses and/or cross-sectional shape and/or cross-sectional area.

In other non-limiting aspects of the present invention, the prosthetic heart valve can include an inner skirt that can be formed of a variety of flexible materials (e.g., polymer [e.g., polyethylene terephthalate (PET), polyester, nylon, Kevlar,®, silicon, etc.], composite material, metal, fabric material, etc.). In one non-limiting embodiment, the material used to partially (e.g., 1-99.999 wt. % and all values and ranges therebetween) or fully form the inner skirt can optionally be substantially non-elastic (i.e., substantially non-stretchable and non-compressible). In another non-limiting embodiment, the material used to partially or fully form the inner skirt can optionally be a stretchable and/or compressible material (e.g., silicone, PTFE, ePTFE, polyurethane, polyolefins, hydrogels, biological materials [e.g., pericardium or biological polymers such as collagen, gelatin, or hyaluronic acid derivatives], etc.). The inner skirt can optionally be formed from a combination of a cloth or fabric material that is coated with a flexible material or with a stretchable and/or compressible material so as to provide additional structural integrity to the inner skirt. The size, configuration, and thickness of the inner skirt is non-limiting (e.g., thickness of 0.1-20 mils and all values and ranges therebetween). The inner skirt can be secured to the inside and/or outside of the frame using various means (e.g., sutures, clamp arrangement, etc.).

In other non-limiting aspects of the present invention, the prosthetic heart valve can optionally include an inner skirt that can be used to 1) at least partially seal and/or prevent paravalvular leakage, 2) at least partially secure the leaflet structure to the frame, 3) at least partially protect the leaflets from damage during the crimping and/or expansion process, and/or 4) at least partially protect the leaflets from damage during the operation of the prosthetic heart valve in the heart.

In other non-limiting aspects of the present invention, the prosthetic heart valve can optionally include an outer or sleeve that is positioned at least partially (e.g. 1-99.999 wt. % and all values and ranges therebetween) about the exterior region of the frame. The outer skirt or sleeve generally is positioned completely around a portion of the outside of the frame. Generally, the outer skirt is positioned about the lower portion of the frame, but does not fully cover the upper half of the frame; however, this is not required. The outer skirt can be connected to the frame by a variety of arrangements (e.g., sutures, adhesive, melted connection, clamping arrangement, etc.). At least a portion of the outer skirt can optionally be located on the interior surface of the frame. Generally, the outer skirt is formed of a more flexible and/or compressible material than the inner skirt; however, this is not required. The outer skirt can be formed of a variety of a stretchable and/or compressible material (e.g., silicone, PTFE, ePTFE, polyurethane, polyolefins, hydrogels, biological materials [e.g., pericardium or biological polymers such as collagen, gelatin, or hyaluronic acid derivatives], etc.). The outer skirt can optionally be formed from a combination of a cloth or fabric material that is coated with the stretchable and/or compressible material to provide additional structural integrity to the outer skirt. The size, configuration, and thickness of the outer skirt is non-limiting. The thickness of the outer skirt is generally 0.1-20 mils (and all values and ranges therebetween).

In other non-limiting aspects of the present invention, the prosthetic heart valve can include a leaflet structure that can be can be attached to the frame and/or skirt. The connection arrangement used to secure the leaflet structures to the frame and/or skirt is non-limiting (e.g., sutures, staples, melted bold, adhesive, clamp arrangement, etc.). The material used to form the leaflet structures include bovine pericardial tissue, biocompatible synthetic materials, or various other suitable natural or synthetic materials.

In other non-limiting aspects of the present invention, the prosthetic heart valve can include a leaflet structure comprised of two or more leaflets (e.g., 2, 3, 4, 5, 6, etc.). In one non-limiting arrangement, the leaflet structure includes three leaflets arranged to collapse in a tricuspid arrangement. The configuration of the leaflet structures is non-limiting.

In other non-limiting aspects of the present invention, the prosthetic heart valve can include a leaflet structure wherein the leaflets of the leaflet structure can optionally be secured to one another at their adjacent sides to form commissures of the leaflet structure (the edges where the leaflets come together). The leaflet structure can be secured together by a variety of connection arrangement (e.g., sutures, adhesive, melted bond, clamping arrangement, etc.).

In other non-limiting aspects of the present invention, the prosthetic heart valve can include a leaflet structure wherein one or more of the leaflets can optionally include reinforcing structures or strips to 1) facilitate in securing the leaflets together, 2) facilitate in securing the leaflets to the skirt and/or frame, and/or 3) inhibit or prevent tearing or other types of damage to the leaflets.

In other non-limiting aspects of the present invention, the frame of the prosthetic heart valve is partially (e.g. 1-99.999 wt. % and all values and ranges therebetween) or fully formed of a metal material that includes a) standard stainless steel, b) standard CoCr alloy or standard MP35N alloy or a standard Phynox alloy or standard Elgiloy alloy or standard L605 alloy, c) standard TiAlV alloy, d) standard aluminum alloy, e) standard nickel alloy, f) standard titanium alloy, g) standard tungsten alloy, h) standard molybdenum alloy, i) standard copper alloy, j) standard beryllium-copper alloy, k) standard Nitinol alloy, l) refractory metal alloy, or m) metal alloy that includes at least 15 atomic weight percent (awt. %) rhenium. As defined herein, a standard stainless-steel alloy includes 10-28 wt. % (weight percent) chromium, 0-35 wt. % nickel, 0-4 wt. % molybdenum, 0-2 wt. % manganese, 0-0.75 wt. % silicon, 0-0.3 wt. % carbon, 0-5 wt. % titanium, 0-10 wt. % niobium, 0-5 wt. % copper, 0-4 wt. % aluminum, 0-10 wt. % tantalum, 0-1 wt. % Se, 0-2 wt. % vanadium, 0-2 wt. % tungsten, and at least 50 wt. % iron. A standard 316L alloy includes 17-19 wt. % chromium, 13-15 wt. % nickel, 2-4 wt. % molybdenum, 2 wt. % max manganese, 0.75 wt. % max silicon, 0.03 wt. % max carbon, balance iron. As defined herein, a standard CoCr alloy includes 15-32 wt. % chromium, 1-36 wt. % nickel, 2-18 wt. % molybdenum, 0-18 wt. % iron, 0-1 wt. % titanium, 0-0.15 wt. % manganese, 0-0.15 wt. % silver, 0-0.025 wt. % carbon, 0-16 wt. % tungsten, 0-2 wt. % Si, 0-2 wt. % aluminum, 0-1 wt. % iron, 30-68 wt. % cobalt. As defined herein, a standard MP35N alloy includes 18-22 wt. % chromium, 32-38 wt. % nickel, 8-12 wt. % molybdenum, 0-2 wt. % iron, 0-0.5 wt. % silicon, 0-0.5 wt. % manganese, 0-0.2 wt. % carbon, 0-2 wt. % titanium, 0-0.1 wt. % phosphorous, 0-0.1 wt. % boron, 0-0.1 wt. % sulfur, 0-0.15 wt. % silver, and balance cobalt. As defined herein, a standard Phynox and standard Elgiloy alloy includes 38-42 wt. % cobalt, 18-22 wt. % chromium, 14-18 wt. % iron, 13-17 wt. % nickel, 6-8 wt. % molybdenum. As defined herein, a standard L605 alloy includes 18-22 wt. % chromium, 14-16 wt. % tungsten, 9-11 wt. % nickel, balance cobalt. As defined herein, a standard TiAlV alloy includes 5.5-6.75 wt. % aluminum, 3.5-4.5 wt. % vanadium, 85-93 wt. % titanium, 0-0.4 wt. % iron, 0-0.2 wt. % carbon. A standard Ti-6Al-4V alloy incudes 3.5-4.5 wt. % vanadium, 5.5-6.75 wt. % aluminum, 0.3 wt. % max iron, 0.2 wt. % max oxygen, 0.08 wt. % max carbon, 0.05 wt. % max nitrogen, 0.015 wt. % max hydrogen H, 0.05 wt. % max yttrium, balance titanium. As defined herein, a standard aluminum alloy includes 80-99 wt. % aluminum, 0-12 wt. % silicon, 0-5 wt. % magnesium, 0-1 wt. % manganese, 0-0.5 wt. % scandium, 0-0.5 wt. % beryllium, 0-0.5 wt. % yttrium, 0-0.5 wt. % cerium, 0-0.5 wt. % chromium, 0-3 wt. % iron, 0-0.5, 0-9 wt. % zinc, 0-0.5 wt. % titanium, 0-3 wt. % lithium, 0-0.5 wt. % silver, 0-0.5 wt. % calcium, 0-0.5 wt. % zirconium, 0-1 wt. % lead, 0-0.5 wt. % cadmium, 0-0.05 wt. % bismuth, 0-1 wt. % nickel, 0-0.2 wt. % vanadium, 0-0.1 wt. % gallium, and 0-7 wt. % copper. As defined herein, a standard nickel alloy includes 30-98 wt. % nickel, 5-25 wt. % chromium, 0-65 wt. % iron, 0-30 wt. % molybdenum, 0-32 wt. % copper, 0-32 wt. % cobalt, 2-2 wt. % aluminum, 0-6 wt. % tantalum, 0-15 wt. % tungsten, 0-5 wt. % titanium, 0-6 wt. % niobium, 0-3 wt. % silicon. As defined herein, a standard titanium alloy includes 80-99 wt. % titanium, 0-6 wt. % aluminum, 0-3 wt. % tin, 0-1 wt. % palladium, 0-8 wt. % vanadium, 0-15 wt. % molybdenum, 0-1 wt. % nickel, 0-0.3 wt. % ruthenium, 0-6 wt. % chromium, 0-4 wt. % zirconium, 0-4 wt. % niobium, 0-1 wt. % silicon, 0.0.5 wt. % cobalt, 0-2 wt. % iron. As defined herein, a standard tungsten alloy includes 85-98 wt. % tungsten, 0-8 wt. % nickel, 0-5 wt. % copper, 0-5 wt. % molybdenum, 0-4 wt. % iron. As defined herein, a standard molybdenum alloy includes 90-99.5 wt. % molybdenum, 0-1 wt. % nickel, 0-1 wt. % titanium, 0-1 wt. % zirconium, 0-30 wt. % tungsten, 0-2 wt. % hafnium, 0-2 wt. % lanthanum. As defined herein, a standard copper alloy includes 55-95 wt. % copper, 0-40 wt. % zinc, 0-10 wt. % tin, 0-10 wt. % lead, 0-1 wt. % iron, 0-5 wt. % silicon, 0-12 wt. % manganese, 0-12 wt. % aluminum, 0-3 wt. % beryllium, 0-1 Wt. % cobalt, 0-20 wt. % nickel. As defined herein, a standard beryllium-copper alloy includes 95-98.5 wt. % copper, 1-4 wt. % beryllium, 0-1 wt. % cobalt, and 0-0.5 wt. % silicon. As defined herein, a standard Nitinol alloy includes 42-58 wt. % nickel and 42-58 wt. % titanium. As defined herein, a refractory metal alloy is a metal alloy that includes at least 20 wt. % of one or more of molybdenum, rhenium, niobium, tantalum or tungsten. Non-limiting refractory metal alloys include MoRe alloy, ReW alloy, MoReCr alloy, MoReTa alloy, MoReTi alloy, WCu alloy, ReCr, molybdenum alloy, rhenium alloy, tungsten alloy, tantalum alloy, niobium alloy, etc.

In other non-limiting aspects of the present invention, the frame of the prosthetic heart valve is formed of a metal material that includes a metal alloy that contains at least 15 awt. % rhenium. It has been found that for several metal alloys the inclusion of at least 15 awt % rhenium results in the ductility and/or tensile strength of the metal alloy to improve as compared to a metal alloy is that absent rhenium. Such improvement in ductility and/or tensile strength due to the inclusion of at least 15 awt. % rhenium in the metal alloy is referred to as the “rhenium effect.” As defined herein, a “rhenium effect” is a) an increase of at least 10% in ductility of the metal alloy caused by the addition of rhenium to the metal alloy, and/or b) an increase of at least 10% in tensile strength of the metal alloy caused by the addition of rhenium to the metal alloy. It has been found for some metal alloys (e.g., standard stainless steel, standard CoCr alloys, standard TiAlV alloys, standard aluminum alloys, standard nickel alloys, standard titanium alloys, standard tungsten alloys, standard molybdenum alloys, standard copper alloys, standard MP35N alloys, standard beryllium-copper alloys, etc.), the inclusion of at least 15 awt. % rhenium results in improved ductility and/or tensile strength. It has been found that the addition of rhenium to a metal alloy can result in the formation of a twining alloy in the metal alloy that results in the overall ductility of the metal alloy to increase as the yield and tensile strength increases as a result of reduction and/or work hardening of the metal alloy that includes the rhenium addition. The rhenium effect has been found to occur when the atomic weight of rhenium in the metal alloy is at least 15% (e.g., 15-99 awt. % rhenium in the metal alloy and all values and ranges therebetween). For example, for standard stainless-steel alloys, the rhenium effect can begin to be present when the stainless steel alloy is modified to include a rhenium amount of at least 5-10 wt. % (and all values and ranges therebetween) of the stainless steel alloy. For standard CoCr alloys, the rhenium effect can begin to be present when the CoCr alloy is modified to include a rhenium amount of at least 4.8-9.5 wt. % (and all values and ranges therebetween) of the CoCr alloy. For standard TiAlV alloys, the rhenium effect can begin to be present when the TiAlV alloy is modified to include a rhenium amount of at least 4.5-9 wt. % (and all values and ranges therebetween) of the TiAlV alloy. At can be appreciated, the rhenium content in the above non-limiting examples can be greater than the minimum amount to create the rhenium effect in the metal alloy.

In other non-limiting aspects of the present invention, the metal alloy includes at least 15 awt. % rhenium, and at least 0.1 wt. % (e.g., 0.1 wt. % to 96 wt. % and all values and ranges therebetween) of one or more of aluminum, boron, beryllium, bismuth, cadmium, calcium, cerium, cerium oxide, chromium, cobalt, copper, gallium, gold, hafnium, iridium, iron, lanthanum, lanthanum oxide, lead, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, yttrium oxide, zinc, zirconium, and/or zirconium oxide, and the metal alloy optionally includes 0-2 wt. % (and all values and ranges therebetween) of a combination of other metals, carbon, oxygen, phosphorous, sulfur, hydrogen and/or nitrogen, and which metal alloy exhibits a rhenium effect. In another non-limiting embodiment, the metal alloy that is used to partially or fully form the frame of the prosthetic heart valve is a refractory metal alloy. In one non-limiting embodiment, the metal alloy that is used to partially or fully form the frame of the prosthetic heart valve is a standard stainless steel alloy that has been modified to include at least 15 awt. % rhenium. In one non-limiting embodiment, the metal alloy that is used to partially or fully form the frame of the prosthetic heart valve is a standard cobalt chromium alloy that has been modified to include at least 15 awt. % rhenium. In one non-limiting embodiment, the metal alloy that is used to partially or fully form the frame of the prosthetic heart valve is a standard TiAlV alloy that has been modified to include at least 15 awt. % rhenium. In one non-limiting embodiment, the metal alloy that is used to partially or fully form the frame of the prosthetic heart valve is a standard aluminum alloy that has been modified to include at least 15 awt. % rhenium. In one non-limiting embodiment, the metal alloy that is used to partially or fully form the frame of the prosthetic heart valve is a standard nickel alloy that has been modified to include at least 15 awt. % rhenium. In one non-limiting embodiment, the metal alloy that is used to partially or fully form the frame of the prosthetic heart valve is a standard titanium alloy that has been modified to include at least 15 awt. % rhenium. In one non-limiting embodiment, the metal alloy that is used to partially or fully form the frame of the prosthetic heart valve is a standard tungsten alloy that has been modified to include at least 15 awt. % rhenium. In one non-limiting embodiment, the metal alloy that is used to partially or fully form the frame of the prosthetic heart valve is a standard molybdenum alloy that has been modified to include at least 15 awt. % rhenium. In one non-limiting embodiment, the metal alloy that is used to partially or fully form the frame of the prosthetic heart valve is a standard copper alloy that has been modified to include at least 15 awt. % rhenium. In one non-limiting embodiment, the metal alloy that is used to partially or fully form the frame of the prosthetic heart valve is a standard beryllium-copper alloy that has been modified to include at least 15 awt. % rhenium.

In other non-limiting aspects of the present invention, the metal alloy that is used to partially or fully form the frame of the prosthetic heart valve includes rhenium and molybdenum, and the weight percent of rhenium in the metal alloy is greater that the weight percent of molybdenum in the metal alloy. In one non-limiting embodiment, the metal alloy includes rhenium and molybdenum, and the weight percent of rhenium in the metal alloy is greater that the weight percent of molybdenum in the metal alloy, and the weight percent of one or more of bismuth, niobium, tantalum, tungsten, titanium, vanadium, chromium, manganese, yttrium, zirconium, technetium, ruthenium, rhodium, hafnium, osmium, copper, and iridium in the metal alloy is greater that the weight percent of molybdenum in the metal alloy, and the metal alloy optionally includes 0-2 wt. % of a combination of other metals, carbon, oxygen, phosphorous, sulfur, hydrogen and/or nitrogen.

In other non-limiting aspects of the present invention, the metal alloy that is used to partially or fully form the frame of the prosthetic heart valve includes rhenium and molybdenum, and the atomic weight percent of rhenium to the atomic weight percent of the combination of one or more of bismuth, niobium, tantalum, tungsten, titanium, vanadium, chromium, manganese, yttrium, zirconium, technetium, ruthenium, rhodium, hafnium, osmium, copper, and iridium is 0.4:1 to 2.5:1 (and all values and ranges therebetween).

In other non-limiting aspects of the present invention, the metal alloy that is used to partially or fully form the frame of the prosthetic heart valve includes at least 15 awt. % rhenium plus at least two metals selected from the group of molybdenum, bismuth, chromium, iridium, niobium, tantalum, titanium, yttrium, and zirconium, and the content of the metal alloy that includes other elements and compounds is 0-0.1 wt. %. In another non-limiting embodiment, the metal alloy includes rhenium, molybdenum, and chromium. In another non-limiting embodiment, the metal alloy includes at least 35 wt. % (e.g., 35-75 wt. % and all values and ranges therebetween) rhenium, and the metal alloy also includes chromium. In one non-limiting embodiment, the metal alloy includes at least 35 wt. % rhenium and at least 25 wt. % (e.g., 25-49.9 wt. % and all values and ranges therebetween) of the metal alloy includes chromium, and optionally 0.1-40 wt. % (and all values and ranges therebetween) of the metal alloy includes one or more of aluminum, bismuth, calcium, carbon, cerium oxide, cobalt, copper, gold, hafnium, iridium, iron, lanthanum, lanthanum oxide, lead, magnesium, manganese, molybdenum, nickel, niobium, osmium, platinum, rare earth metals, rhodium, ruthenium, silver, tantalum, technetium, titanium, tungsten, vanadium, yttrium, yttrium oxide, zinc, zirconium, and/or zirconium oxide, and optionally 0-2 wt. % of a combination of other metals, carbon, oxygen, phosphorous, sulfur, hydrogen and/or nitrogen. In another non-limiting embodiment, the metal alloy includes 15-50 awt. % rhenium (and all values and ranges therebetween) and 0.5-70 awt. % chromium (and all values and ranges therebetween). In another non-limiting embodiment, the metal alloy includes 15-50 awt. % rhenium (and all values and ranges therebetween) and 0.5-70 awt. % tantalum (and all values and ranges therebetween). In another non-limiting embodiment, the metal alloy includes 15-50 awt. % rhenium (and all values and ranges therebetween) and 0.5-70 awt. % niobium (and all values and ranges therebetween). In another non-limiting embodiment, the metal alloy includes 15-50 awt. % rhenium (and all values and ranges therebetween) and 0.5-70 awt. % titanium (and all values and ranges therebetween). In another non-limiting embodiment, the metal alloy includes 15-50 awt. % rhenium (and all values and ranges therebetween) and 0.5-70 awt. % zirconium (and all values and ranges therebetween). In another non-limiting embodiment, the metal alloy includes 15-50 awt. % rhenium (and all values and ranges therebetween) and 0.5-70 awt. % molybdenum (and all values and ranges therebetween). In another non-limiting embodiment, the metal alloy includes at least 15 awt. % rhenium, greater than 50 wt. % titanium (e.g., 51-80 wt. % and all values and ranges therebetween), 15-45 wt. % (and all values and ranges therebetween) niobium, 0-10 wt. % (and all values and ranges therebetween) zirconium, 0-15 wt. % (and all values and ranges therebetween) tantalum, and 0-8 wt. % molybdenum (and all values and ranges therebetween).

In other non-limiting aspects of the present invention, the prosthetic heart valve can include, contain, and/or be coated with one or more agents that facilitate in the success of the prosthetic heart valve and/or treated area. The term “agent” includes, but is not limited to a substance, pharmaceutical, biologic, veterinary product, drug, and analogs or derivatives otherwise formulated and/or designed to prevent, inhibit and/or treat one or more clinical and/or biological events, and/or to promote healing. The amount of agent included on, in, and/or used in conjunction with prosthetic heart valve (when the agent is used) is about 0.01-100 ug per mm² (and all values and ranges wherein between) and/or at least about 0.00001 wt. % of device; however, other amounts can be used. The amount of two of more agents on, in, and/or used in conjunction with prosthetic heart valve can be the same or different. The one or more agents can be coated on and/or impregnated in prosthetic heart valve by a variety of mechanisms such as, but not limited to, spraying (e.g., atomizing spray techniques, etc.), flame spray coating, powder deposition, dip coating, flow coating, dip-spin coating, roll coating (direct and reverse), sonication, brushing, plasma deposition, depositing by vapor deposition, MEMS technology, and rotating mold deposition. The amount of two of more agents on, in, and/or used in conjunction with prosthetic heart valve (when two one more agents are used) can be the same or different.

In other non-limiting aspects of the present invention, the one or more agents on and/or in the prosthetic heart valve (when used on the prosthetic heart valve) can optionally be released in a controlled manner so the area in question to be treated is provided with the desired dosage of agent over a sustained period of time. As can be appreciated, controlled release of one or more agents on the prosthetic heart valve is not always required and/or desirable.

In other non-limiting aspects of the present invention, the one or more polymers used to at least partially control the release of one or more agents from the prosthetic heart valve can be porous or non-porous.

In other non-limiting aspects of the present invention, different agents can optionally be located in and/or between different polymer coating layers and/or on the structure of the prosthetic heart valve. As can also be appreciated, many other and/or additional coating combinations and/or configurations can be used. The concentration of one or more agents, the type of polymer, the type and/or shape of internal structures in the prosthetic heart valve, and/or the coating thickness of the one or more agents can be used to control the release time, the release rate and/or the dosage amount of one or more agents; however, other or additional combinations can be used. The one or more agents and/or polymers can be coated on the prosthetic heart valve by a variety of mechanisms such as, but not limited to, spraying (e.g., atomizing spray techniques, etc.), dip coating, roll coating, sonication, brushing, plasma deposition, and/or depositing by vapor deposition.

In other non-limiting aspects of the present invention, the thickness of each polymer layer and/or layer of agent is generally at least about 0.01 μm and is generally less than about 150 μm (e.g., 0.01-149.9999 μm and all values and ranges therebetween). In one non-limiting embodiment, the thickness of a polymer layer and/or layer of agent is about 0.02-75 μm, more particularly about 0.05-50 μm, and even more particularly about 1-30 μm. As can be appreciated, other thicknesses can be used.

In other non-limiting aspects of the present invention, a variety of polymers can be coated on the prosthetic heart valve and/or used to form at least a portion of the prosthetic heart valve. When one or more layers of polymer are coated onto at least a portion of the prosthetic heart valve, the one or more coatings can be applied by a variety of techniques such as, but not limited to, vapor and/or plasma deposition, spraying, dip-coating, roll coating, sonication, atomization, brushing, and/or the like; however, other or additional coating techniques can be used.

In other non-limiting aspects of the present invention, one or more portions of the prosthetic heart valve can optionally 1) include the same or different agents, 2) include the same or different amount of one or more agents, 3) include the same or different polymer coatings, 4) include the same or different coating thicknesses of one or more polymer coatings, 5) have one or more portions of the prosthetic heart valve controllably release and/or uncontrollably release one or more agents, and/or 6) have one or more portions of the prosthetic heart valve controllably release one or more agents and one or more portions of the prosthetic heart valve uncontrollably release one or more agents.

In other non-limiting aspects of the present invention, the prosthetic heart valve can optionally include a marker material that facilitates enabling the prosthetic heart valve to be properly positioned at the treatment site. The marker material is typically designed to be visible to electromagnetic waves (e.g., x-rays, microwaves, visible light, infrared waves, ultraviolet waves, etc.); sound waves (e.g., ultrasound waves, etc.); magnetic waves (e.g., MRI, etc.); and/or other types of electromagnetic waves (e.g., microwaves, visible light, infrared waves, ultraviolet waves, etc.). In one non-limiting embodiment, the marker material is visible to x-rays (i.e., radiopaque). The marker material can form all or a portion of the prosthetic heart valve and/or be coated on one or more portions (flaring portion and/or body portion, at ends of prosthetic heart valve, at or near transition of body portion and flaring section, etc.) of the prosthetic heart valve. The location of the marker material can be on one or multiple locations on the prosthetic heart valve. The size of the one or more regions including the marker material can be the same or different. The marker material can be spaced at defined distances from one another to form ruler-like markings on the prosthetic heart valve to facilitate in the positioning of the prosthetic heart valve in a body passageway. The marker material can be a rigid or flexible material.

In other non-limiting aspects of the present invention, the prosthetic heart valve can optionally include one or more micro-structures (e.g., micro-needle, micro-pore, micro-cylinder, micro-cone, micro-pyramid, micro-tube, micro-parallelepiped, micro-prism, micro-hemisphere, teeth, rib, ridge, ratchet, hinge, zipper, zip-tie-like structure, etc.) on the surface of the prosthetic heart valve. As defined herein, a “micro-structure” is a structure having at least one dimension (e.g., average width, average diameter, average height, average length, average depth, etc.) that is no more than about 2 mm, and typically no more than about 1 mm.

In other non-limiting aspects of the present invention, the medical device, handle, and/or prosthetic heart valve can be form by one or more manufacturing processes. These manufacturing processes can include, but are not limited to, laser cutting, etching, annealing, drawing, pilfering, electroplating, electro-polishing, machining, plasma coating, 3D printed coatings, 3D printing, chemical vapor deposition, chemical polishing, cleaning, pickling, ion beam deposition or implantation, sputter coating, vacuum deposition, etc. In one non-limiting embodiment, at least a portion or all of the medical device, handle, and/or prosthetic heart valve is formed by a 3D printing process.

In other non-limiting aspects of the present invention, one or more components of the prosthetic heart valve (e.g., frame, inner skirt, outer skirt, leaflets, material used to secure leaflets to frame, etc.) can be partially (e.g., 1% to 99.99% and all values and ranges therebetween) or fully be coated with an enhancement coating to improve one or more properties of the prosthetic heat valve (e.g., change exterior color of material having coated surface, increase surface hardness by use of the coated surface, increase surface toughness material having coated surface, reduced friction via use of the coated surface, improve scratch resistance of material that has the coated surface, improve impact wear of coated surface, improve resistance to corrosion and oxidation of coated material, form a non-stick coated surface, improve biocompatibility of material having the coated surface, reduce toxicity of material having the coated surface, reduce ion release from material having the coated surface, the enhancement coating forms a surface that is less of an irritant to cell about the coated surface after the prosthetic heart valve is implanted, reduces the rate to which cells grown on coated surface after prosthetic heart valve is implanted, reduce rate to which leaflets fail to properly operate after prosthetic heart valve is implanted, promote production and/or release of NO, etc.). In one non-limiting embodiment, only the frame of the prosthetic heart valve includes the enhancement coating, and wherein the frame is partially (e.g., 1-99.99% and all values and ranges therebetween) or fully coated with the enhancement coating. In another non-limiting embodiment, only one or more of all of the leaflets of the prosthetic heart valve include the enhancement coating, and wherein one or more or all of the leaflets are partially (e.g., 1-99.99% and all values and ranges therebetween) or fully coated with the enhancement coating. In another non-limiting embodiment, only the inner skirt of the prosthetic heart valve includes the enhancement coating, and wherein the inner skirt is partially (e.g., 1-99.99% and all values and ranges therebetween) or fully coated with the enhancement coating. In another non-limiting embodiment, only the outer skirt of the prosthetic heart valve includes the enhancement coating, and wherein the outer skirt is partially (e.g., 1-99.99% and all values and ranges therebetween) or fully coated with the enhancement coating. In another non-limiting embodiment, two or more or all of a) the frame, b) one or more or all of the leaflets, c) the inner skirt and d) the outer skirt of the prosthetic heart valve are partially (e.g., 1-99.99% and all values and ranges therebetween) or are fully coated with the enhancement coating. Non-limiting enhancement coatings that can be applied to a portion or all of the outer surface of one or more components of the prosthetic heart valve includes chromium nitride (CrN), diamond-like carbon (DLC), titanium nitride (TiN), titanium oxynitride or titanium nitride oxide (TiNOx), zirconium nitride (ZrN), zirconium oxide (ZrO₂), zirconium oxynitride (ZnNxOy) [e.g., cubic ZrN:O, cubic ZrO₂:N, tetragonal ZrO₂:N, and monoclinic ZrO₂:N phase coatings], oxyzirconium-nitrogen-carbon (ZrNC), zirconium OxyCarbide (ZrOC), and combinations of such coatings. In one non-limiting embodiment, the one or more enhancement coatings are optionally applied to a portion or all of the outer surface of one or more components of the prosthetic heart valve by a vacuum process using an energy source to vaporize material and deposit a thin layer of enhancement coating material. Such vacuum coating process, when used, can include a physical vapor deposition (PVD) process (e.g., sputter deposition, cathodic arc deposition or electron beam heating, etc.), chemical vapor deposition (CVD) process, atomic layer deposition (ALD) process, or a plasma-enhanced chemical vapor deposition (PE-CVD) process. In another non-limiting embodiment, the thickness of the enhancement coating is greater than 1 nanometer (e.g., 2 nanometers to 100 microns and all values and ranges therebetween), and typically 0.1-25 microns, and more typically 0.2-10 microns.

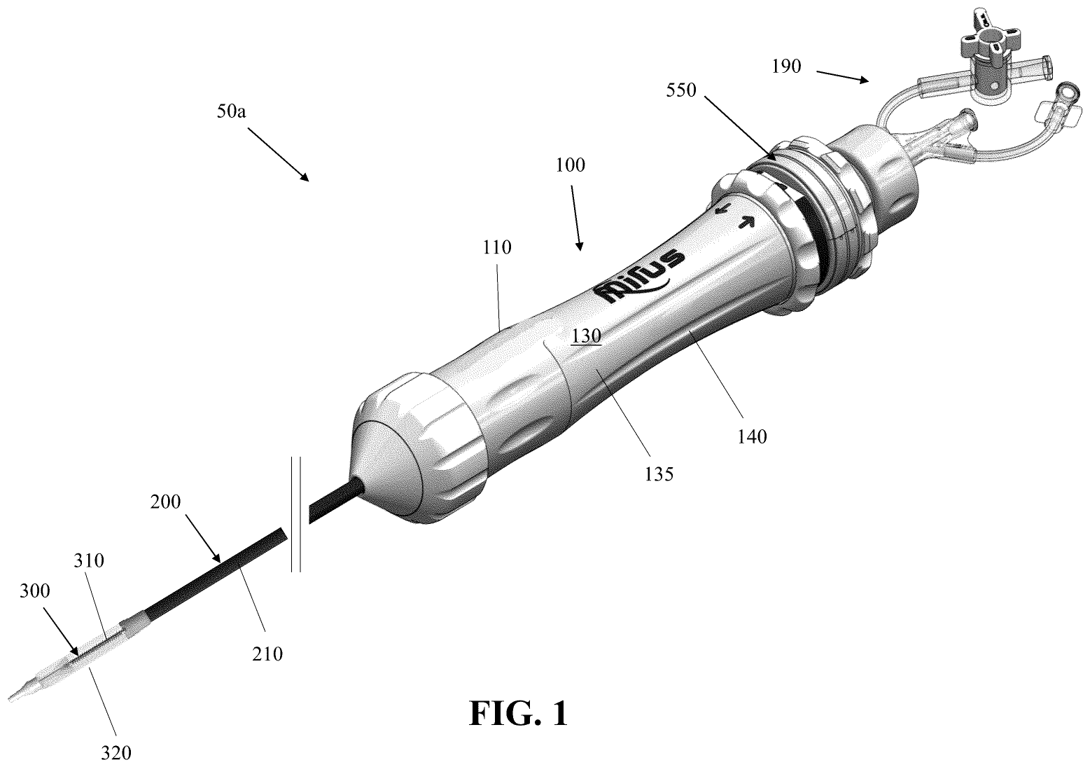

One or more implementations of the subject application will now be described with reference to the attached FIGS. 1-4, wherein like reference numerals are used to refer to like elements throughout.

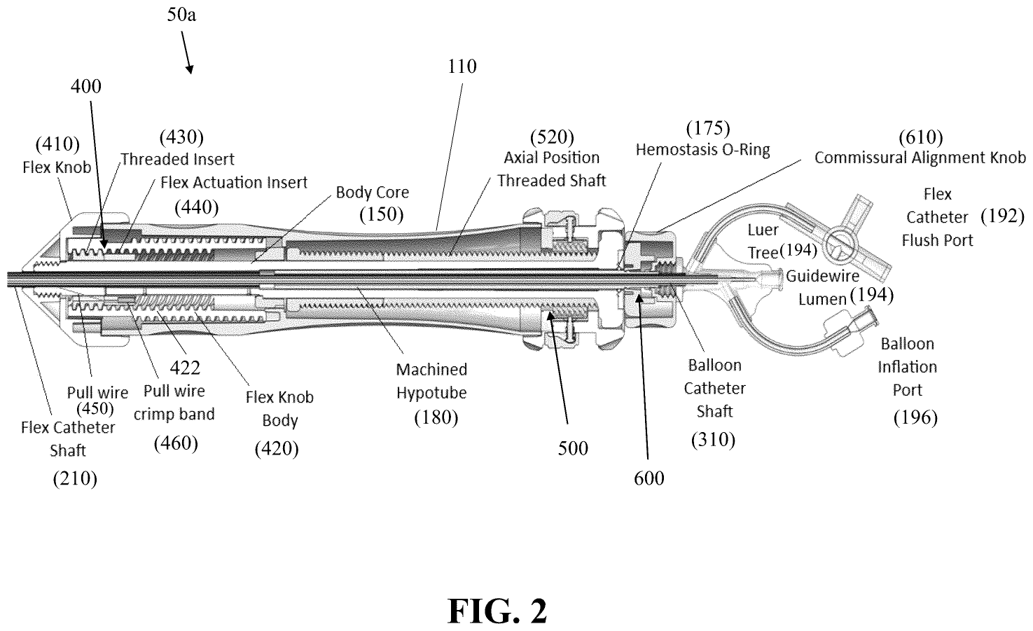

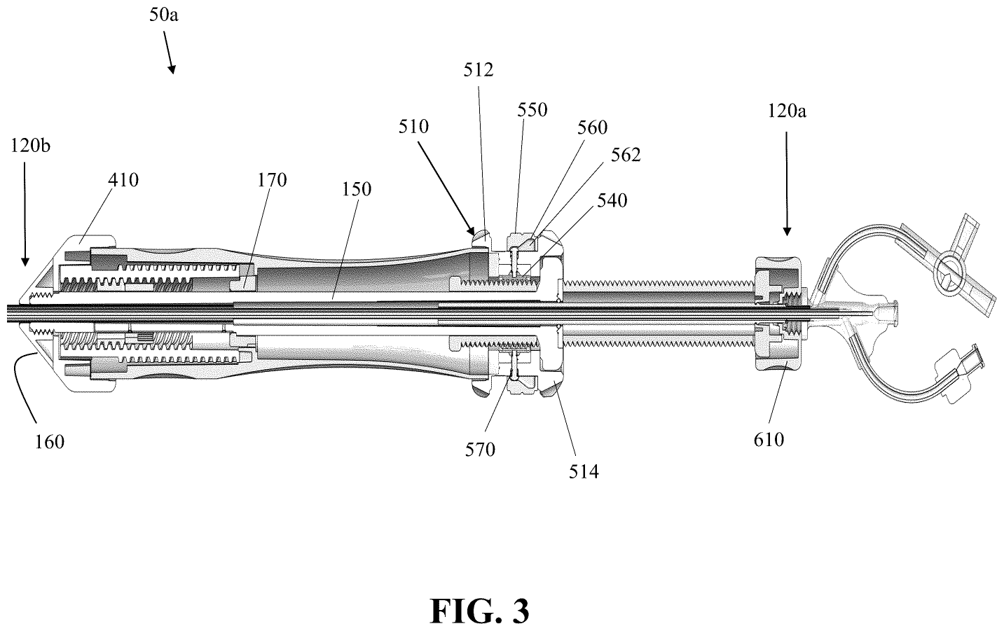

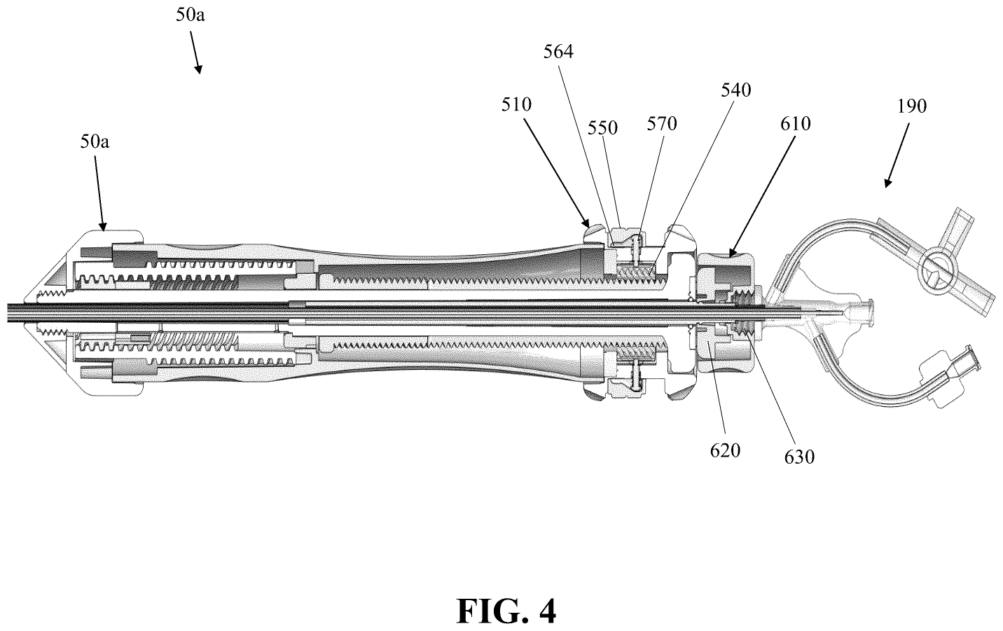

FIG. 1 depicts an exemplary, non-limiting delivery system 50a comprising a non-limiting handle arrangement 100. FIG. 2 is a cross-sectional side view of the delivery handle arrangement 100 of FIG. 1. FIG. 3 is a cross-sectional side view of the delivery handle arrangement 100 of FIG. 1, wherein the delivery handle arrangement 100 is configured in a locked position. FIG. 4 is a cross-sectional side view of the delivery handle arrangement 100 of FIG. 1, wherein the delivery handle arrangement 100 is configured in an unlocked position.

With reference to FIGS. 1-4, there is provided a delivery system 50a that is configured for use with a prosthetic heart valve. The delivery system 50a includes a delivery handle arrangement 100, a flex catheter or similar flexible guide catheter 200 having a shaft 210, and a balloon catheter 300 disposed coaxially within the flex catheter 200. The balloon catheter 300 includes a shaft 310 and an inflatable balloon 320. The delivery handle arrangement 100 includes an elongated housing 110 having a proximal end 120a and a distal end 120b. The housing 110 is a generally circular cross-sectional shape along a longitudinal axis of the housing 110; however, other shapes can be used. The cross-sectional area of the housing 110 can optionally vary along the longitudinal length of the housing 110. For example, in the present non-limiting embodiment, the housing 110 comprises a smaller cross-sectional area in the mid-region of the housing 110, as compared to the regions located at and/or near the proximal and distal ends 120a, 120b. In the present non-limiting embodiment, the housing 110 has a length that is sufficient for a user to easily grasp the housing 110 with the user's hands (e.g. 4-12 inches and all values and ranges therebetween). The housing 110 further comprises an outer handle 135 having an outer surface 130, wherein the outer surface 130 includes one or more ribs 140 formed thereon that are configured to facilitate in the gripping of the outer handle 135.

With specific reference to FIGS. 2-4, the delivery handle arrangement 100 includes a hollow, internal body core 150 that forms the center of the delivery handle arrangement 100 and lies along the central longitudinal axis of the delivery handle arrangement 100 between the proximal end 120a and the distal end 120b. The outer surface of the flex catheter shaft 210 is fixed to the inner surface of the body core 150. In one non-limiting aspect of the present disclosure, the flex catheter shaft 210 is fixed to the body core 150 with an adhesive; however, other connection arrangements can be used (e.g., melted connection, mechanical connection, etc.). The balloon catheter 300 and shaft 310 extend coaxially through the flex catheter shaft 210. The flex catheter 200 and the balloon catheter 300 exit the distal end 120b of the housing 110.

Referring back to FIGS. 1-4, the delivery handle arrangement 100 further includes a flex mechanism 400, an adjustment mechanism 500, and/or a commissure alignment (CA) mechanism or similar rotational mechanism 600. The flex mechanism 400 is located near the distal end 120b of the housing 110, and the adjustment mechanism 500 and the CA mechanism 600 are located near the proximal end 120a of the housing 110. The flex mechanism 400 includes a rotatable knob 410 that is connected to a knob body 420 that is contained within the housing 110. The knob body 420 includes a plurality of female threads 422 that receive and engage a threaded insert 430. The flex mechanism 400 further includes a flex actuation insert 440, a pull wire 450 that is threaded through an opening or lumen on one side of the flex catheter shaft 210, and a crimp band 460 that is secured within the flex actuation insert 440. The pull wire 450 is coupled to the crimp band 460. When the knob 410 is rotated in a first direction (either clockwise or counterclockwise), the threaded insert 430 and the flex actuation insert 440 translate axially along the knob body 420, thereby pulling the wire 450 and causing the flex catheter shaft 210 to flex or displace by one or more predetermined angles.

In one non-limiting example of the present invention, rotating the knob 410 in a first direction (either clockwise or counterclockwise) causes the threaded insert 430 and the flex actuation insert 440 to translate along the knob body 420 from the distal end 120b towards the proximal end 120a, thereby pulling the wire 450 and causing the flex catheter shaft 210 to flex or displace by a predetermined angle.

Still referring to FIGS. 1-4, the adjustment mechanism 500 includes a control knob 510 having a first portion 512, a second portion 514, and a plurality of latching threads 540. The adjustment mechanism 500 further includes an axial position threaded shaft 520 that is contained within the housing 110. The adjustment mechanism 500 is configured to advance or retract the balloon catheter shaft 310 relative to the flex catheter 200. The adjustment mechanism 500 is transitionable between a locked position (FIG. 3) and an unlocked position (FIG. 4) by way of a switch 550. The switch 550 is positioned between the first portion 512 and the second portion 514 of the control knob 510. The switch 550 comprises a channel 560 formed therein and a catch 570, wherein the catch 570 is moveable within the channel 560. The channel 560 includes a first stop 562 and a second stop 564, wherein the first stop 562 has a greater width than that of the second stop 564. In the present non-limiting embodiment, the switch 550 and the catch 570 have a substantially circular cross-sectional shape.

Still referring to FIGS. 1-4, when the adjustment mechanism 500 is in the locked position: (i) the catch 570 is positioned at the second stop 564 of the channel 560; (ii) the switch 550 is positioned closer to the second portion 514 of the control knob 510, and (iii) the catch 570 is engaged with the latching threads 540. In this locked position, the relative axial position between the balloon catheter shaft 310 and the flex catheter 200 is fixed, and the balloon catheter shaft 310 axial position can be adjusted in a controlled manner by rotating (either clockwise or counterclockwise) the control knob 510. Rotating the control knob 510 causes the latching threads 540 to engage the axial position threaded shaft 520, thereby causing the axial position threaded shaft 520 to translate linearly along the longitudinal axis of the delivery handle arrangement 100.

In one non-limiting example of the present invention, when in the locked position, rotating the control knob 510 in a first direction (either clockwise or counterclockwise) causes the axial position threaded shaft 520 to translate linearly from the proximal end 120a towards the distal end 120b, thereby advancing the balloon catheter shaft 310 relative to the flex catheter 200. In another non-limiting example of the present invention, when in the locked position, rotating the control knob in a second direction (either clockwise or counterclockwise) causes the axial position threaded shaft 520 to translate linearly from the distal end 120b towards the proximal end 120a, thereby retracting the balloon catheter shaft 310 relative to the flex catheter 200.

Still referring to FIGS. 1-4, when the adjustment mechanism 500 is in the unlocked position: (i) the catch 570 is positioned at the first stop 562 of the channel 560; (ii) the switch 550 is positioned closer to the first portion 512 of the control knob 510; and (iii) the catch 570 is disengaged from the latching threads 540. In this unlocked position, the user can freely advance and retract the balloon catheter shaft 310 within the flex catheter 200 by manually pushing (e.g. to advance) or pulling (e.g. to retract) the alignment knob 610 and/or the luer tree 190 (discussed in greater detail below).

In one non-limiting example of the present invention, when in the unlocked position, pushing the alignment knob 610 and/or the luer tree 190 causes the axial position threaded shaft 520 to translate linearly from the proximal end 120a towards the distal end 120b, thereby advancing the balloon catheter shaft 310 relative to the flex catheter 200. In another non-limiting example of the present invention, when in the unlocked position, pulling the alignment knob 610 and/or the luer tree 190 causes the axial position threaded shaft 520 to translate linearly from the distal end 120b towards the proximal end 120a, thereby retracting the balloon catheter shaft 310 relative to the flex catheter 200.

Still referring to FIGS. 1-4, the CA mechanism 600 includes an alignment knob 610 having an inner cap 620 and a gear 630. The alignment knob 610 is coupled to the axial position threaded shaft 520. The alignment knob 610 is rotatable (either clockwise or counterclockwise) around the gear 630 to control the rotational movement of the balloon catheter shaft 310 relative to the flex catheter 200. The rotation of the alignment knob 610 correspondingly rotates the axial position threaded shaft 520. Rotation of the balloon catheter shaft 310 correspondingly rotates the crimped valve thereon, thereby controlling the rotational orientation of the implant leaflet attachment points (commissure) relative to the native anatomical features. The alignment knob 610 can be rotated to control the rotation movement of the balloon catheter shaft 310 regardless of the locked or unlocked position of the adjustment mechanism 500.

Still referring to FIGS. 1-4, a luer tree 190 is coupled to the alignment knob 610. The luer tree 190 includes a flex catheter flush port 192, a guidewire lumen 194 that is configured to receive a guidewire, and a balloon inflation port 196.

Still referring to FIGS. 1-4, the housing 110 further includes a mid-ring 170, a hemostasis O-ring 175, and a hypotube 180. The mid-ring 170 is contained within the housing 110 and is configured to maintain the outer handle 135 in a stationary, non-rotatable position relative to the rotatable components (e.g. knob 410, control knob 510, alignment knob 610, etc.) of the delivery handle arrangement 100. The hypotube 180 is contained within the body core 150 and is configured to allow flush fluid to flow over the balloon catheter shaft 310 and into the flex catheter shaft 210 as the prosthetic heart valve is moved and/or expanded at the treatment site. The hypotube 180 is free to translate linearly and rotationally relative to the body core 150 as the axial position threaded shaft 520 is correspondingly translated linearly (via the adjustment mechanism 500) and/or rotationally (via the CA mechanism 600). The hemostasis O-ring 175 creates a sliding hemostatic seal between the inner surface of the body core 150 and an outer surface of the hypotube 180.

Still referring to FIGS. 1-4, the housing 110 further includes a nose cone 160 attached to the body core 150 about the distal end 120b. The nose cone 160 is configured to hold the knob 410 and the knob body 420 in place relative to the delivery handle arrangement 100.

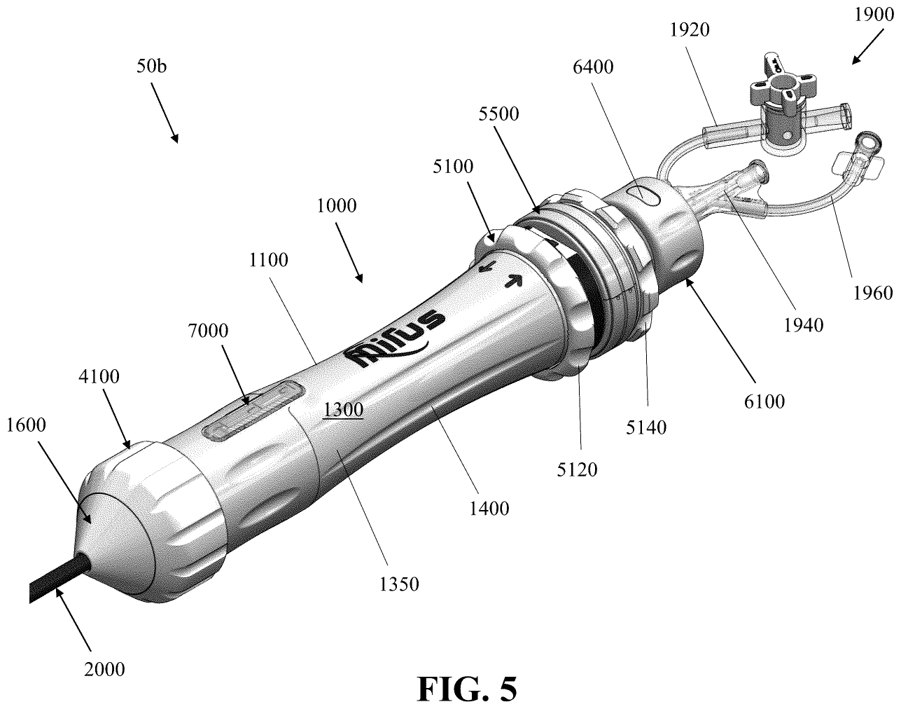

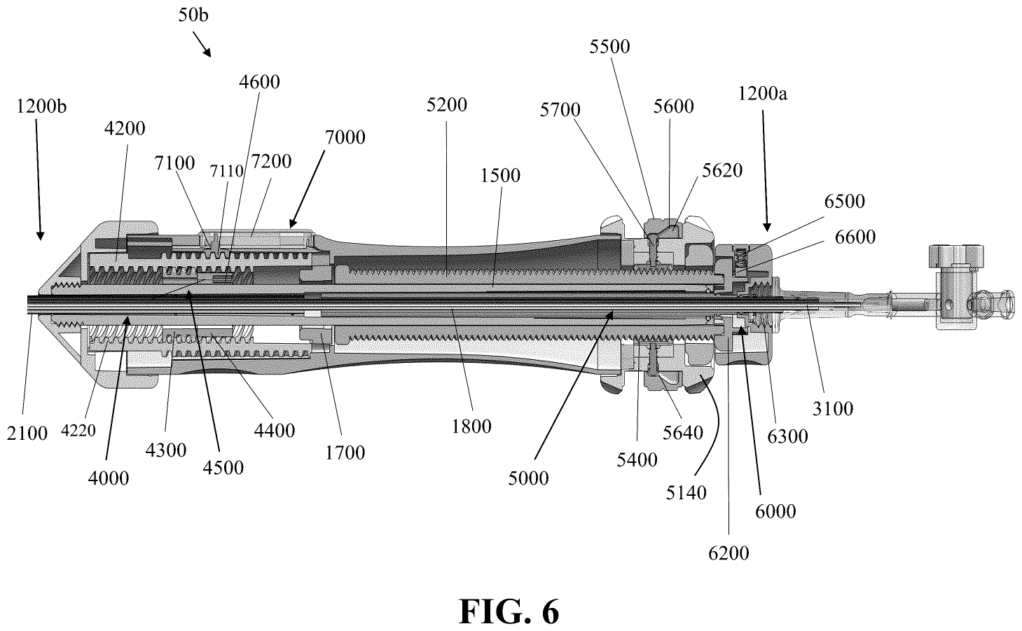

One or more implementations of the subject application will now be described with reference to the attached FIGS. 5-6, wherein like reference numerals are used to refer to like elements throughout. FIG. 5 depicts another exemplary, non-limiting delivery system 50b comprising a non-limiting handle arrangement 1000. FIG. 6 is a cross-sectional side view of the delivery handle arrangement 1000 of FIG. 5.

With reference to FIGS. 5-6, there is provided a delivery system 50b comprising a delivery handle arrangement 1000. The delivery system 50b and the handle 1000 function similarly to the delivery system 50a and the handle 100 and include similar components, the differences being that the delivery system 50b includes: (i) a flex indicator 7000 that is configured to provide the degree or amount of flex present in the flex catheter shaft 2100 to a user; and (ii) a cap 6400, a spring 6500, and a tooth 6600 that are included in a commissure alignment (CA) mechanism 6000.

It is to be appreciated that any of the disclosed components incorporated in the delivery system 50a could be used in combination with the disclosed components of the delivery system 50b. It is also to be appreciated that any of the disclosed components incorporated in the delivery system 50b could be used in combination with the disclosed components of the delivery system 50a.

Still referring to FIGS. 5-6, there is provided a delivery system 50b that is configured for use with a prosthetic heart valve, the delivery system 50b having a delivery handle arrangement 1000 and a flex catheter or similar flexible guide catheter 2000 having a shaft 2100. A balloon catheter shaft 3100 is disposed coaxially within the flex catheter 2000. The delivery handle arrangement 1000 includes an elongated housing 1100 having a proximal end 1200a and a distal end 1200b. The housing 1100 is a generally circular cross-sectional shape along a longitudinal axis of the housing 1100. The cross-sectional area of the housing 1100 can optionally vary along the longitudinal length of the housing 1100. For example, in the present non-limiting embodiment, the housing 1100 comprises a smaller cross-sectional area in the mid-region of the housing 1100, as compared to the regions located at and/or near the proximal and distal ends 1200a, 1200b. In the present non-limiting embodiment, the housing 1100 has a length that is sufficient for a user to easily grasp the housing 1100 with the user's hands (e.g. 4-12 inches and all values and ranges therebetween). The housing 1100 further comprises an outer handle 1350 having an outer surface 1300, wherein the outer surface 1300 includes one or more ribs 1400 formed thereon that are configured to facilitate in the gripping of the outer handle 1350.

The delivery handle arrangement 1000 includes a hollow, internal body core 1500 that forms the center of the delivery handle arrangement 1000 and lies along the central longitudinal axis of the delivery handle arrangement 1000 between the proximal end 1200a and the distal end 1200b. The outer surface of the flex catheter shaft 2100 is fixed to the inner surface of the body core 1500. In one non-limiting aspect of the present disclosure, the flex catheter shaft 2100 is fixed to the body core 1500 with an adhesive. The balloon catheter shaft 3100 extends coaxially through the flex catheter shaft 2100. The flex catheter 2000 and the balloon catheter shaft 3100 exit the distal end 1200b of the housing 1100.

Still referring to FIGS. 5-6, the delivery handle arrangement 1000 further comprises a flex mechanism 4000, an adjustment mechanism 5000, and a commissure alignment (CA) mechanism or similar rotational mechanism 6000. The flex mechanism 4000 is located near the distal end 1200b of the housing 1100, and the adjustment mechanism 5000 and the CA mechanism 6000 are located near the proximal end 1200a of the housing 1100. The flex mechanism 4000 includes a rotatable knob 4100 that is connected to a knob body 4200 that is contained within the housing 1100. The knob body 4200 includes a plurality of female threads 4220 that receive and engage a threaded insert 4300. The flex mechanism 4000 further includes a flex actuation insert 4400, a pull wire 4500 that is threaded through an opening or lumen on one side of the flex catheter shaft 2100, and a crimp band 4600 that is secured within the flex actuation insert 4400. The pull wire 4500 is coupled to the crimp band 4600. When the knob 4100 is rotated in a first direction (either clockwise or counterclockwise), the threaded insert 4300 and the flex actuation insert 4400 translate axially along the knob body 4200, thereby pulling the wire 4500 and causing the flex catheter shaft 2100 to flex or displace by a predetermined angle.

In In one non-limiting example of the present invention, rotating the knob 4100 in a first direction (either clockwise or counterclockwise) causes the threaded insert 4300 and the flex actuation insert 4400 to translate along the knob body 4200 from the distal end 120b towards the proximal end 120a, thereby pulling the wire 4500 and causing the flex catheter shaft 2100 to flex or displace by a predetermined angle.

In the present non-limiting embodiment, the delivery handle arrangement 1000 includes a flex indicator 7000. The flex indicator 7000 includes a needle 7100 and a protector 7200. The flex indicator 7000 is configured to provide the degree or amount of flex present in the flex catheter shaft 2100 to a user. The needle 7100 includes one or more threads 7110 that are configured to engage the knob body 4200. As the knob 4100 is rotated (either clockwise or counterclockwise), the needle 7100 translates along the knob body 4200, indicating the degree or amount of flex present in the flex catheter shaft 2100 to a user.

Still referring to FIGS. 5-6, the adjustment mechanism 5000 includes a control knob 5100 having a first portion 5120, a second portion 5140, and a plurality of latching threads 5400. The adjustment mechanism 5000 further includes an axial position threaded shaft 5200 that is contained within the housing 1100. The adjustment mechanism 5000 is configured to advance or retract the balloon catheter shaft 3100 relative to the flex catheter 2000. The adjustment mechanism 5000 is transitionable between a locked position and an unlocked position by way of a switch 5500. The switch 5500 is positioned between the first portion 5120 and the second portion 5140 of the control knob 5100. The switch 5500 comprises a channel 5600 formed therein and a catch 5700, wherein the catch 5700 is moveable within the channel 5600. The channel 5600 includes a first stop 5620 and a second stop 5640, wherein the first stop 5620 has a greater width than that of the second stop 5640. In the present non-limiting embodiment, the switch 5500 and the catch 5700 have a substantially circular cross-sectional shape.

When the adjustment mechanism 5000 is in the locked position (see FIG. 6): (i) the catch 5700 is positioned at the second stop 5640 of the channel 5600; (ii) the switch 5500 is positioned closer to the second portion 5140 of the control knob 5100; and (iii) the catch 5700 is engaged with the latching threads 5400. In this locked position, the relative axial position between the balloon catheter shaft 3100 and the flex catheter 2000 is fixed, and the balloon catheter shaft 3100 axial position can be adjusted in a controlled manner by rotating (either clockwise or counterclockwise) the control knob 5100. Rotating the control knob 5100 causes the latching threads 5400 to engage the axial position threaded shaft 5200, thereby causing the axial position threaded shaft 5200 to translate linearly along the longitudinal axis of the delivery handle arrangement 1000.

When the adjustment mechanism 5000 is in the unlocked position: (i) the catch 5700 is positioned at the first stop 5620 of the channel 5600; (ii) the switch 5500 is positioned closer to the first portion 5120 of the control knob 5100; and (iii) the catch 5700 is disengaged from the latching threads 5400. In this unlocked position, the user can freely advance and retract the balloon catheter shaft 3100 within the flex catheter 2000 by manually pushing (e.g. to advance) or pulling (e.g. to retract) the alignment knob 6100 and/or the luer tree 1900 (discussed in greater detail below).

Still referring to FIGS. 5-6, the CA mechanism 6000 includes an alignment knob 6100 having an inner cap 6200 and a gear 6300. The alignment knob 6100 is coupled to the axial position threaded shaft 5200. The alignment knob 6100 is rotatable (either clockwise or counterclockwise) around the gear 6300 to control the rotational movement of the balloon catheter shaft 3100 relative to the flex catheter 2000. The rotation of the alignment knob 6100 correspondingly rotates the axial position threaded shaft 5200. Rotation of the balloon catheter shaft 3100 correspondingly rotates the crimped valve thereon, thereby controlling the rotational orientation of the implant leaflet attachment points (commissure) relative to the native anatomical features. The alignment knob 6100 can be rotated to control the rotation movement of the balloon catheter shaft 3100 regardless of the locked or unlocked position of the adjustment mechanism 5000.

The alignment knob 6100 further comprises a cap 6400, a spring 6500, and a tooth 6600. The cap 6400 holds the spring 6500 in place against the tooth 6600. The tooth 660 moves or slides up and down as the tooth 660 rotates around the gear 6300.

Still referring to FIGS. 5-6, a luer tree 1900 is coupled to the alignment knob 6100. The luer tree 1900 includes a flex catheter flush port 1920, a guidewire lumen 1940 that is configured to receive a guidewire, and a balloon inflation port 1960.

The housing 1100 further includes a mid-ring 1700 and a hypotube 1800. The mid-ring 1700 is contained within the housing 1100 and is configured to maintain the outer handle 1350 in a stationary, non-rotatable position relative to the rotatable components (e.g. knob 4100, control knob 5100, alignment knob 6100, etc.) of the delivery handle arrangement 1000. The hypotube 1800 is contained within the body core 1500 and is configured to allow flush fluid to flow over the balloon catheter shaft 3100 and into the flex catheter shaft 2100 as the prosthetic heart valve is moved and/or expanded at the treatment site. The hypotube 1800 is free to translate linearly and rotationally relative to the body core 1500 as the axial position threaded shaft 5200 is correspondingly translated linearly (via the adjustment mechanism 5000) and/or rotationally (via the CA mechanism 6000).

Still referring to FIGS. 5-6, the housing 1100 further includes a nose cone 1600 attached to the body core 1500 about the distal end 1200b. The nose cone 1600 is configured to hold the knob 4100 and the knob body 4200 in place relative to the delivery handle arrangement 1000.

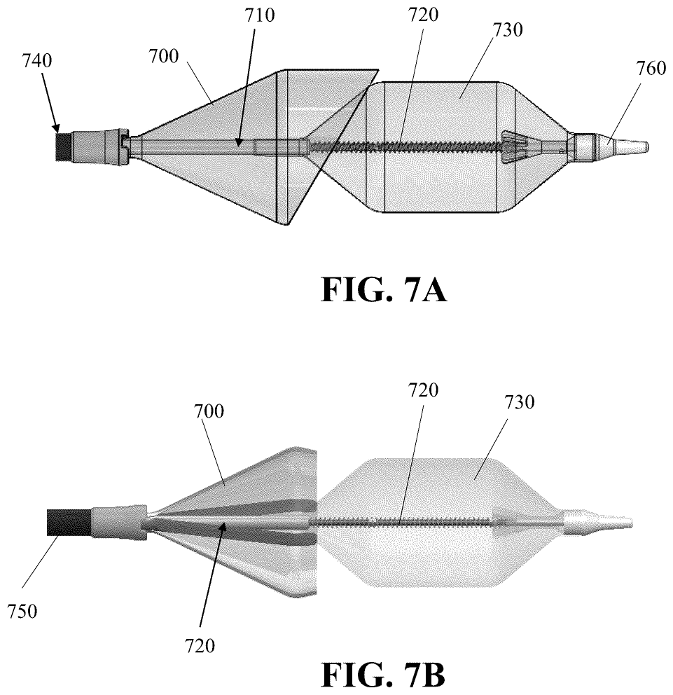

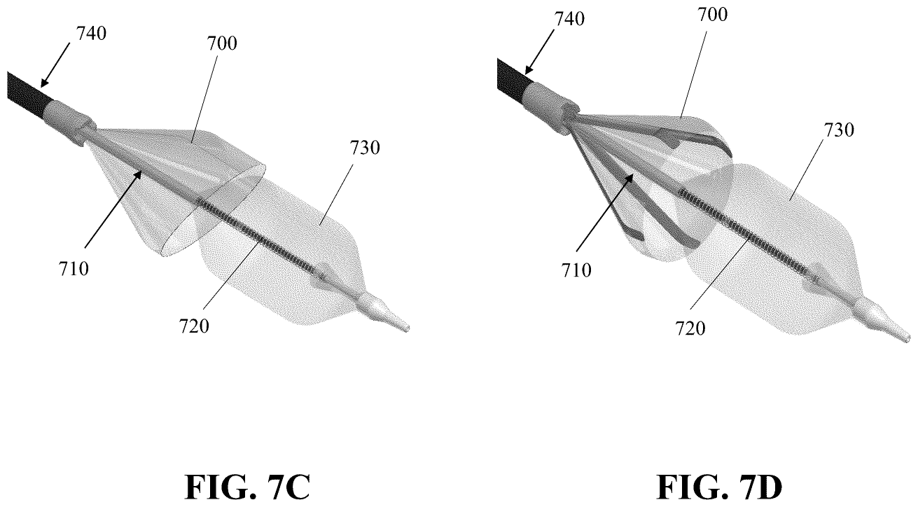

One or more implementations of the subject application will now be described with reference to the attached FIGS. 7A-7D, wherein like reference numerals are used to refer to like elements throughout. FIGS. 7A-7D depict an exemplary, non-limiting embolic capture filter membrane sock or similar filter 700 that is configured for use with a non-limiting flex catheter 740 and a non-limiting balloon catheter 720.

With reference to FIGS. 7A-7D, there is provided an embolic capture filter membrane sock or similar filter 700 that is mounted on the exterior of a balloon catheter shaft 710. Balloon catheter 720 is contained within the balloon catheter shaft 710 and includes an inflatable balloon 730. The balloon catheter shaft 710 extends coaxially within a flex catheter 740 and a flex catheter shaft 750. The balloon catheter 720 further includes a nose 760 that is configured to facilitate movement through arteries or similar conduits.

Still referring to FIGS. 7A-7D, the embolic capture filter membrane sock 700 includes a porous membrane having one or more pores that are sized to effectively block passage of embolic particles (e.g. 50 um) and include sufficient hole density to provide minimal resistance to flow when deployed in the aorta (e.g. 20-60% open area vs. remaining material). When the flex catheter shaft 750 is retracted prior to inflation of the balloon 730 to deploy the prosthetic heart valve, the embolic capture filter membrane sock 700 expands and contacts the inner lumen of the aorta proximal to the balloon 730. When the flex catheter shaft 750 is re-advanced, the embolic capture filter membrane sock 700 closes, thereby containing any trapped emboli. The embolic capture filter membrane sock 700 is opened only during the short timeframe immediately prior to balloon 730 inflation and is closed soon after deflation of the balloon 730. It is to be appreciated that the disclosed embolic capture filter membrane sock 700 can be used with the delivery systems 50a and/or 50b discussed herein.

It should be understood, however, that all of these and similar terms are to be associated with the appropriate physical quantities and are merely convenient labels applied to these quantities.

It will thus be seen that the objects set forth above, among those made apparent from the preceding description, are efficiently attained, and since certain changes may be made in the constructions set forth without departing from the spirit and scope of the disclosure, it is intended that all matter contained in the above description and shown in the accompanying drawings shall be interpreted as illustrative and not in a limiting sense. The disclosure has been described with reference to preferred and alternate embodiments. Modifications and alterations will become apparent to those skilled in the art upon reading and understanding the detailed discussion of the disclosure provided herein. This disclosure is intended to include all such modifications and alterations insofar as they come within the scope of the present disclosure. It is also to be understood that the following claims are intended to cover all of the generic and specific features of the disclosure herein described and all statements of the scope of the disclosure, which, as a matter of language, might be said to fall there between. These and other modifications of the preferred embodiments as well as other embodiments of the disclosure will be obvious from the disclosure herein, whereby the foregoing descriptive matter is to be interpreted merely as illustrative of the disclosure and not as a limitation. It is intended to include all such modifications and alterations insofar as they come within the scope of the appended claims.

The description of embodiments and examples has been presented for purposes of illustration and description. It is not intended to be exhaustive or limiting to the forms described. Numerous modifications are possible in light of the teachings herein. Some of those modifications have been discussed, and others will be understood by those skilled in the art. The embodiments were chosen and described in order to illustrate principles of various embodiments as are suited to particular uses contemplated. The scope is, of course, not limited to the examples set forth herein, but can be employed in any number of applications and equivalent devices by those of ordinary skill in the art.

To aid the Patent Office and any readers of this application and any resulting patent in interpreting the claims appended hereto, applicants do not intend any of the appended claims or claim elements to invoke 35 U.S.C. 112(f) unless the words “means for” or “step for” are explicitly used in the particular claim.

What is claimed:

1. A delivery handle arrangement that facilitates a delivery of an expandable medical device to a treatment site; said delivery handle arrangement comprising:
a housing having an inner core that at least partially extends linearly between a proximal end and a distal end of said housing; and wherein said inner core at least partially receives a flexible catheter and a balloon catheter that is at least partially disposed coaxially within said flexible catheter;
an adjustment mechanism that is configured to control a position of said balloon catheter relative to said flexible catheter; said adjustment mechanism includes:
a control knob that has a connection arrangement; and
a shaft that is configured to translate linearly within said housing; and
a switch that is configured to lock or unlocked said adjustment mechanism.

2. The delivery handle arrangement as defined in claim 1, further comprising a flex mechanism that is configured to bend said flexible catheter at one or more predetermined angles; said flex mechanism includes:
a flex knob that is coupled to a body, and wherein said body is at least partially contained within said housing;
a insert that is engaged with said body;
an actuation insert that has a crimp band; and
a wire member that is coupled to said flexible catheter and said crimp band.

3. The delivery handle arrangement as defined in claim 2, wherein rotation of said flex knob in a first direction causes said insert and said actuation insert to translate linearly along said body from said distal end towards said proximal end, and wherein said wire member pulls said flexible catheter to bend said flexible catheter to said one or more predetermined angles.

4. The delivery handle arrangement as defined in claim 3, further comprising a flex indicator that has a needle, and wherein said needle translates along said body to indicate to a user a degree of flex present in said flexible catheter.

5. The delivery handle arrangement as defined in claim 1, further comprising a rotational mechanism that is configured to rotate said balloon catheter; said rotational mechanism including:
an alignment knob that is coupled to said shaft of said adjustment mechanism; and
a gear; and
wherein rotation of said alignment knob in a first direction causes said balloon catheter to rotate relative to said flexible catheter.

6. The delivery handle arrangement as defined in claim 5, wherein said control knob includes a first portion and a second portion, and wherein said switch is positioned between said first portion and said second portion.

7. The delivery handle arrangement as defined in claim 6, wherein said switch includes:
a channel having a first stop and a second stop, and wherein said first stop has a different width than said second stop; and
a catch that is moveable within said channel.

8. The delivery handle arrangement as defined in claim 7, wherein said switch has a substantially circular cross-sectional shape.

9. The delivery handle arrangement as defined in claim 7, wherein when said adjustment mechanism is locked, a) said catch is positioned at said second stop of the channel, b) said switch is positioned near said second portion of said control knob, and c) said catch is engaged with said engagement arrangement.

10. The delivery handle arrangement as defined in claim 9, wherein rotation of said control knob in a first direction causes said engagement arrangement to engage said shaft and translate said shaft linearly from or near said proximal end towards said distal end within said housing, and wherein rotation of said control knob in a second direction causes said shaft to translate linearly from or near said distal end towards said proximal end.

11. The delivery handle arrangement as defined in claim 7, wherein when the adjustment mechanism is unlocked, a) said catch is positioned at said first stop of said channel, b) said switch is positioned near said first portion of said control knob, and c) said catch is disengaged from said engagement arrangement.

12. The delivery handle arrangement as defined in claim 11, wherein said alignment knob of said rotational mechanism is pushed to advance said shaft towards said distal end, and wherein said alignment knob is pulled to retreat said shaft towards said proximal end.

13. The delivery handle arrangement as defined in claim 5, further comprising a luer tree that is coupled to said alignment knob; said luer tree includes one or more of a flush port, a guidewire lumen that is configured to receive a guidewire, and/or a balloon inflation port.

14. The delivery handle arrangement as defined in claim 1, further comprising:
a hypotube that is contained within said inner core, and wherein said hypotube is configured to allow flush fluid to flow at least partially over said balloon catheter and at least partially into said flexible catheter; and
a sealing arrangement that creates a sliding hemostatic seal between an inner surface of said inner core and an outer surface of said hypotube.

15. The delivery handle arrangement as defined in claim 1, wherein said expandable medical device is a prosthetic heart valve.

16. A system that delivers an expandable medical device to a treatment site, the system comprising:
a flexible catheter;
a balloon catheter that is disposed coaxially within said flexible catheter; and
a delivery handle arrangement; said delivery handle arrangement including:
a housing that has an inner core that extends linearly between a proximal end and a distal end of said housing, and wherein said inner core at least partially receives said flexible catheter and said balloon catheter;
an adjustment mechanism that is configured to control a position of said balloon catheter relative to said flexible catheter, and wherein said adjustment mechanism includes:
a control knob that has an engagement arrangement; and
a shaft that is configured to translate linearly within said housing; and
a switch that is configured to lock or unlocked said adjustment mechanism, and wherein said switch includes:
a channel that has a first stop and a second stop, and wherein said first stop has a different width than the second stop; and
a catch that is moveable within said channel.

17. The system as defined in claim 16, wherein said delivery handle arrangement further includes a flex mechanism that is configured to bend said flexible catheter at one or more predetermined angles; said flex mechanism includes:
a flex knob that is coupled to a body, and wherein said body is at least partially contained within said housing;
an insert that is engaged with said body;
an actuation insert that has a crimp band; and
a wire member that is coupled to said flexible catheter and said crimp band; and
wherein rotation of said flex knob in a first direction causes said insert and said actuation insert to translate linearly along said body from or near said distal end towards said proximal end, and wherein said wire member pulls said flexible catheter to bend said flexible catheter said one or more predetermined angles.

18. The system as defined in claim 16, wherein said delivery handle arrangement further includes a rotational mechanism that is configured to rotate said balloon catheter; said rotational mechanism includes:
an alignment knob that is coupled to said shaft of said adjustment mechanism; and
a gear; and
wherein rotation of said alignment knob in a first direction causes said balloon catheter to rotate relative to said flexible catheter.

19. The system as defined in claim 16, wherein when said adjustment mechanism is locked, then a) said catch is positioned at said second stop of said channel, and b) said catch is engaged with said engagement arrangement; and wherein rotation of said control knob in a first direction causes said engagement arrangement to engage said shaft and translate said shaft linearly from or near said proximal end towards said distal end within said housing, and wherein rotation of said control knob in a second direction causes said shaft to translate linearly from or near said distal end towards said proximal end.

20. The system as defined in claim 18, wherein when said adjustment mechanism is unlocked, then a) said catch is positioned at said first stop of the channel; and b) said catch is disengaged from said engagement arrangement; and wherein said alignment knob of said rotational mechanism is pushed to advance said shaft towards said distal end, and wherein said alignment knob is pulled to retreat said shaft towards said proximal end.
